(19) United States
(12) Patent Application Publication
(10) Pub. No.: US 2014/0121216 A1
(43) Pub. Date: May 1, 2014

(54) INDOLEAMINE DERIVATIVES FOR THE TREATMENT OF CENTRAL NERVOUS SYSTEM DISEASES

(71) Applicants: Marcin Kolaczkowski, Wieliczka (PL); Monika Marcinkowska, Krakow (PL); Adam Bucki, Proszowice (PL); Maciej Pawlowski, Wieliczka (PL); Grzegorz Kazek, Sosnowiec (PL); Marek Bednarski, Krakow (PL); Anna Wesolowska, Krakow (PL)

(72) Inventors: Marcin Kolaczkowski, Wieliczka (PL); Monika Marcinkowska, Krakow (PL); Adam Bucki, Proszowice (PL); Maciej Pawlowski, Wieliczka (PL); Grzegorz Kazek, Sosnowiec (PL); Marek Bednarski, Krakow (PL); Anna Wesolowska, Krakow (PL)

(73) Assignee: Adamed Sp. z o.o., Czosnó k/Warszawy (PL)

(21) Appl. No.: 14/125,750

(22) PCT Filed: Jun. 28, 2012
(86) PCT No.: PCT/IB2012/053309

§ 371 Date: Dec. 12, 2013

(30) Foreign Application Priority Data
Jun. 29, 2011 (PL) P.395469

(51) Int. Cl. C07D 401/12; C07D 209/08

(52) U.S. Cl. CPC C07D 401/12; C07D 209/08
USPC 514/253.07; 544/373; 514/254.09; 544/363

(57) ABSTRACT

Indoleamine derivatives of formula (IA), R¹ represents benzyl unsubstituted or substituted with halogen atom, —OH, or C₁-C₃-alkyl; phenylsulphonyl unsubstituted or substituted in the phenyl ring with halogen atom, —OH or C₁-C₃-alkyl; G¹ represents phenoxyalkyl, heteroaryloxyalkyl- or heterocyclyloxyalkyl-piperazine moiety; and pharmaceutically acceptable salts and solvates thereof.

The compounds may be useful for the treatment and/or prevention of the central nervous system disorders.

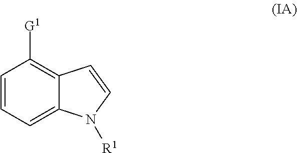

### FIELD OF THE INVENTION

The present invention relates to novel indoleamine derivatives having affinity to dopaminergic, serotoninergic and adrenergic receptors as well as to serotonine transporter receptors, method for manufacturing thereof, pharmaceutical compositions containing the same and to the use thereof. The compounds may be useful for the treatment of diseases of the central nervous system (CNS), such as schizophrenia, bipolar affective disorder, depression, anxiety disorders, sleep disorders or Alzheimer disease.

### STATE OF ART

CNS disorders are considered a global medical problem. A number of people suffering from those diseases constantly grows, particularly in highly developed countries and intensively developing ones.

Among all psychiatric diseases, schizophrenia, bipolar affective disorder, depression, anxiety, sleep disorders and addictions are the major ones. The main neurologic disorders are Alzheimer's disease, Parkinson's disease, epilepsy and different pain disorders.

Antipsychotic drugs, which are main treatment of schizophrenia, are divided into two main classes on the basis of their liability to induce neurological side effects after long-term treatment. Typical antipsychotic drugs, such as chlorpromazine and haloperidol, induce after repeated administration various extrapyramidal side effects (EPS) including Parkinson-like symptoms and tardive dyskinesia. Repeated treatment with so called atypical antipsychotic drugs, such as clozapine, risperidone, olanzapine, quetiapine, ziprasidone and aripiprazole, is associated with a lower incidence of neurological side effects. Typical antipsychotics reduce positive symptoms but do not reduce negative symptoms and cognitive dysfunctions. Plasma prolactin levels are increased in humans, and there is a gain in body weight potentially leading to the development of metabolic syndrome. Atypical antipsychotic drugs effectively reduce positive symptoms and also to some extent negative symptoms and cognitive disturbances producing less serious EPS. Atypical antipsychotic drugs differ in their propensity to elevate plasma prolactin levels in humans. Typical antipsychotic drugs block dopamine D2 receptors in the mesolimbic and nigrostriatal system. This mechanism is responsible for the antipsychotic effect (reduction of positive symptoms) as well as induction of EPS. Clinical support for the dopamine hypothesis of antipsychotic drug action was provided by PET findings of high dopamine D2 receptor occupancy in the striatum of patients responding to different antipsychotic drug treatments. Patients with a good response show dopamine D2 receptor occupancy of more than 65% (Nord M, Farde L. Antipsychotic occupancy of dopamine receptors in schizophrenia. CNS Neuroscience & Therapeutics. 2010; 17:97.). The occurrence of EPS seems to be related to a higher occupancy of dopamine D2 receptors (above 80%). Atypical antipsychotics, also called second generation antipsychotic drugs, have clinical approvals for the treatment of psychosis and mania. Each drug has a unique pharmacodynamic and pharmacokinetic profile. Some of atypical antipsychotic drugs have additional antidepressant, anxiolytic or hypnotic profile (Schwartz T. L., Stahl S. M., CNS Neurosci. Ther.; 17(2), 110-7, 2011). Atypical antipsychotic drugs have in common a potent serotonin 5-HT2A receptor antagonism in relation to a weaker dopamine D2 receptor antagonism. This pharmacodynamic property is the basis of “atypicality” (Meltzer H. Y., Neuropsychopharmacology; 1, 193-6, 1989). Antagonism of 5-HT2A receptors likely allows more dopamine activity and neurotransmission to occur in the nigrostriatal system to avoid EPS. The same mechanism may allow small improvement in negative symptoms, and 5-HT2 antagonism in the tuberoinfundibular pathway may help to avoid hyperprolactinemia (Schwartz T. L., Stahl S. M., CNS Neurosci. Ther.; 17(2), 110-7, 2011).

Dopaminergic D2 receptors are the primary biological target of antipsychotic therapy. It is a recognized fact that that blockade of these receptors in the mesolimbic system is responsible for the antipsychotic activity of neuroleptics, in particular for preventing the positive symptoms. All antipsychotic drugs currently used reveal at least moderate affinity for dopamine D2 receptors. However, blockade of these receptors in the nigrostriatal system if not compensated by a partial agonism to these receptors or by affecting other receptors (5-HT2A, 5-HT1A, alfa2c), may be a cause of extrapyramidal disorders, such as drug-induced parkinsonism, and within tuberoinfundibular pathway—of hyperprolactinaemia (Miyamoto S. et al., Mol. Psychiatry; 10(1), 79-104, 2005).

Dopaminergic D3 receptors are localized in limbic cortex and thus a preferential blockade of these receptors offers locally selective antidopaminergic activity. This results in increased effectiveness in reducing positive symptoms of schizophrenia sparing the blockade of extrapyramidal system and therefore reduces the risk of the main side effect such as pseudoparkinson's syndrome. Moreover, several preclinical data suggests that D3 dopamine receptor antagonism is more efficient in reducing the negative symptoms of schizophrenia and improves working memory. (Gray, J. A., Roth B. L.; Schizophr. Bull.; 33(5, 1100-19, 2007).

Serotoninergic neurons interact with dopaminergic neurons. Antagonistic activity of antipsychotics against serotoninergic receptors 5-HT2A type can stimulate the release of dopamine in the extrapyramidal, tuberoinfundibular systems and prefrontal cortex but not in the limbic system, what can result in alleviation of undesirable extrapyramidal symptoms and hyperprolactinaemia induced by D2 receptor blockade and in increased effectiveness of the drug against some of the negative symptoms of schizophrenia, without increasing the positive symptoms. It is considered that high affinity for 5-HT2A receptors, higher than for D2 receptors, is one of the reasons of atypicality of the second-generation antipsychotics. Similar effects to those caused by the blockade of 5-HT2A receptors, are achieved by stimulation of serotonin receptor type 5-HT1A (aripiprazole, ziprasidone). It is assumed that stimulation of 5-HT1A receptors takes part in the antipsychotic effect in combination with D2 receptor blockade, especially in the safety profile of drug as well as is beneficial in fighting mood and cognitive symptoms of schizophrenia (Kim D. et al., Neurotherapeutics, 6(1), 78-85, 2009).

Despite the advances that have been made in the development of antidepressants, there are clearly still unmet clinical needs with respect to both efficacy and side effects. These needs range from efficacy in treatment of resistant patients (about 30%) to improve onset, to reductions in side effects such as sexual dysfunction, gastrointestinal events, sedation, weight gain. There are multiple approaches to improve current pharmacological means of modulating biogenic amines neurotransmission by either combining mechanisms or alternatively selectively stimulating/blocking receptor subtypes that may trigger improved efficacy or fewer side effects. One of them is combination therapies that maintain the benefits associated with selective serotonin reuptake inhibitors (SSRIs) (blockers of serotonin transporter) but attempt to either improve efficacy or reduce side effects by adding additional mechanism involving blockade of 5-HT2A or 5-HT2C receptors (Milian M., Neurotherapeutics, 6(1), 53-77, 2009). 5-HT2A receptor antagonists administered alone may produce antidepressant activity and also co-administered with SSRIs augment their antidepressant effects. The mechanism for this interaction may be a further increase in extracellular serotonin levels produced when SSRIs are given with 5-HT2A antagonists. Moreover, blockade of 5-HT2A receptors is part of the pharmacological profile of antidepressant drugs such as mianserin and mirtazapine.

Serotoninergic receptors type 5-HT6 are almost exclusively localized in the central nervous system (CNS). Both the localization of the 5-HT6 receptors in limbic and cortical brain areas and relatively potent affinity and antagonistic activity of several antipsychotics (clozapine, olanzapine, sertindole) and antidepressants (mianserin, amitryptiline) at 5-HT6 receptors are suggestive of a potential role in pathophysiology and treatment of CNS disorders. Recent data in the literature indicate that blockade of 5-HT6 receptors may be implicated in a pro-cognitive effect due to the increase in cholinergic transmission, in antidepressant activity due to the increase in noradrenergic and dopaminergic one, as well as in an anxiolytic effect. It is evident that the 5-HT6 receptor has emerged as a very interesting molecular target and antagonists of this receptor may serve as potential drugs in treatment of disorders characterized by cognitive impairments, such as Alzheimer's disease, schizophrenia, depression, anxiety (Liu K., Robichaud A., Drug Development Research 70,145-168, 2009; Wesotowska, A; Nikiforuk, A, Neuropharmacology 52(5), 1274-83, 2007). Moreover, 5-HT6 receptor antagonists have been demonstrated to be active in reduction of food intake and body weight by clinically approved mechanism that is consistent with an enhancement of to satiety. Hence, several compounds with 5-HT6 receptor antagonistic activity are currently being clinically evaluated for the treatment of obesity (Heal D. et al., Pharmacology therapeutics, 117(2), 207-231, 2008).

Intensive research conducted since 1993 indicates that serotoninergic 5-HT7 receptors may play some role in the control of circadian rhythms, sleep, thermoregulation, cognitive processes, pain and migraine, as well as in neuronal excitability. Potent affinity and antagonistic activity of several antipsychotic and antidepressant drugs at 5-HT7 receptors suggest a potential role of these receptors in the pathophysiology of many neuropsychiatric disorders. Taking account of the behavioral data presented in the literature, it has been established that selective 5-HT7 receptor antagonists produce antidepressant and anxiolytic activity in rats and mice (Wesolowska A. et al., Neuropharmacology 51, 578-586, 2006). Using mouse models of antipsychotic activity, Galici et al. showed that selective 5-HT7 receptor antagonist SB-269970 may also evoke antipsychotic-like effects (Galici R. et al., Behav. Pharmacol.; 19(2), 153-9, 2008).

Serotoninergic 5-HT2C and histaminergic H1 receptors localized in hypothalamus play important role in food intake regulation. Blockade of both types of these receptors produced by antipsychotic drugs is most closely correlated with increased risk of weight gain and diabetes. On the other hand, blockade of 5-HT2C receptors, mostly localized in cortical areas and in the hippocampus, striatum, septal nuclei, thalamic and midbrain nuclei, may produce profitable antidepressant and pro-cognitive effects. In the substantia nigra, 5-HT2C receptors are co-localised with GABA, indicating that they yield indirect control of dopaminergic transmission. Consequently, the blockade of 5-HT2C receptors, together with the 5-HT2A receptor one, would potentiate the D2 receptor-mediated tonic inhibitory control of dopaminergic projection, with protective effect against extrapyramidal symptoms (Kim D. et al., Neurotherapeutics, 6(1), 78-85, 2009). Histaminergic H1 receptor blockade produced by antipsychotic drugs may be implicated in sedative effect that is clinically profitable in controlling arousal accompanies the acute phase of psychosis. It seems that simultaneous reduction in affinity of new molecule for both types of these receptors may be an element that protects against excessive body weight. However, the total elimination of affinity for these receptors may not be necessary because of certain benefits of blockade of 5-HT2C and H1 receptors.

Blockade of alpha2 adrenergic receptors potentiates antidepressants-induced increase of extracellular monoamines. This may suggest that substances inhibiting monoamine transporters and simultaneously blocking alpha2 adrenergic receptors may be potent and fast acting new antidepressants. Moreover, alpha2 antagonists potentiate acetylcholine secretion in the frontal cortex and may improve cognitive functions, what may provide additional advantages both in antidepressant therapy and antipsychotic therapy (especially improvement in negative symptoms). Blockade of alpha2 adrenergic receptors may also counteract sexual dysfunctions caused by serotonin reuptake inhibitors (Millan M., Neurotherapeutics, 6(1), 53-77, 2009). Alpha2 antagonists may also be beneficial in reducing extrapyramidal symptoms caused by blockade of D2 receptors in the striatum. Similarly, blockade of alpha1 adrenergic receptors, despite potential peripheral adverse effects involving hypotension, may cause some central nervous system benefits involving decrease in the risk of extrapyramidal side effects caused be antipsychotics. This may be associated with interaction between noradrenergic and serotoninergic neurons (Horacek J. et al., CNS Drugs, 20(5), 389-409, 2006).

Sigma receptors are a separate group of CNS receptors; however their physiological role is still unknown. It has been shown that some psychotomimetic substances like phencyclidine, metamphetamine, heroin or dextrometorphan are potent sigma receptor agonists. On the other hand, a classic antipsychotic drug, haloperidol, is a strong antagonist of sigma receptors, what may be important for its antipsychotic potential. It has been established that selective sigma receptor agonists may produce antidepressant effect (Cobos E. et al., Curr. Neuropharmacol., 6(4), 344-66, 2008). The above findings provide evidence that sigma receptors affinity may contribute to the overall beneficial pharmacological profile of a new psychotropic drug.

Because of important role of cholinergic system in the cognitive processes, current research is focused on substances which can directly or indirectly potentiate the activity of cholinergic system. This includes substances which are agonists of selected subtypes of nicotinic or muscarinic receptors and antagonists of 5-HT6 receptors. On the other hand, potential procognitive effects evoked by interaction with the above receptors may be masked by cholinolytic activity. Thus, in the scope of interest are substances free of antagonistic properties against cholinergic receptors. Moreover this strategy allows elimination of many undesired peripheral autonomic effects like constipations, dry mouth or tachycardia (Miyamoto S. et al., Mol. Psychiatry; 10(1), 79-104, 2005). In addition, it has been found that M3 muscarinic receptors are engaged in the control of insulin secretion, and their activation stimulates pancreas to secrete insulin. Hence, it can be expected that M3 receptors blockade may be unfavorable in terms of the risk of development of type II diabetes in patients treated with second generation antipsychotics (for ex. olanzapine, clozapine, quetiapine). Recent research is focused on substances free of this undesired effect (Silvestre J. S., Prous J., Methods Find. Exp. Clin. Pharmacol.; 27(5), 289-304, 2005).

Another serious side effects caused by antipsychotic drugs, e.g. sertindole, ziprasidone, are cardiac arrhythmias associated with delayed repolarization of cardiomyocytes. This condition appears on electrocardiograms (ECG) as prolonged corrected QT interval (QTc), what is most often evoked by substances which block hERG potassium channels. To prevent introduction to the developmental pipelines drugs with pro-arrhythmic potential, at a very early stage of research new substances are screened in vitro for their potency to block hERG potassium channels, using electrophysiological methods (Recanatini M. et al., Med. Res. Rev., 25(2), 133-66, 2005).

Although introduction of new psychotropic drugs (among others neuroleptics, antidepressants, benzodiazepines, acetylocholinesterase inhibitors) since 50-thies of the XX century was an unquestioned breakthrough, therapy of neuropsychiatric disorders is still far from satisfactory both because of limited efficacy and wide spectrum of side effects caused by available drugs. These disadvantages are a challenge for modern pharmacotherapy and there is a continuous effort to search for new, more effective psychotropic drugs.

From the state of art there are known certain indolepiperazine derivatives.

In WO99/05140 indole and 2,3-dihydroindole derivatives having antagonistic activity towards 5-HT1A serotoninergic receptors and inhibiting serotonine reuptake are described.

In WO99/55672 hydroxyalkyl derivatives of alicyclic amines showing antagonistic activity towards D2 dopaminergic receptors, as well as antagonistic and agonistic activity towards 5-HT1A receptors.

WO2004/046124 relates to benzoxazinone derivatives having high affinity for 5-HT1 receptors and/or ability to inhibit serotonin reuptake.

In WO02/32863 indole derivatives having antagonistic activity towards 5-HT6 receptors are disclosed.

Certain indoleamine derivatives having activity towards D4 and 5-HT1A and/or 5-HT2A receptors and/or serotonin reuptake inhibition are disclosed in WO98/28293.

In EP900792A and WO97/36893 compounds of high affinity for D2 dopaminergic and 5-HT1A serotoninergic receptors are described.

In WO96/03400 indolopiperazine derivatives being potent agonists and antagonists of 5-HT1 serotoninergic receptors are disclosed.

WO01/49680 discloses aminoindole derivatives, potently binding to 5-HT1A receptors and useful for the treatment of certain psychiatric and neurological disorders.

In WO02/365621-aryl and 1-alkylsulphonyl heterocyciylbenzazoles as 5-HT6 receptor ligands are described.

Aim of the Invention

The aim of the present invention is to provide novel compounds potentially useful for the treatment of diseases of the central nervous system. A further aim of the invention is to provide novel compounds useful for the treatment of diseases of central nervous system having higher effectiveness compared to currently used medicaments. Yet further aim of the present invention is to provide novel compounds useful for the treatment of diseases of the central nervous system, which could allow to eliminate or minimize adverse effects associated with currently used therapies.

### DISCLOSURE OF THE INVENTION

The present invention relates to novel indoleamine compounds having the structure represented by the general formula (IA)

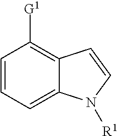

wherein
R¹ represents benzyl unsubstituted or substituted with halogen atom, —OH, or C₁-C₃-alkyl; or
phenylsulphonyl unsubstituted or substituted in the phenyl ring with halogen atom, —OH or C₁-C₃-alkyl;
G¹ represents piperazine moiety of the following formula

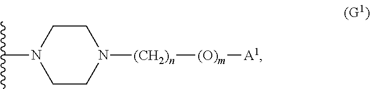

wherein
n is 3 or 4,
m is 1,
A¹ represents phenyl unsubstituted or substituted with one substituent selected from the group consisting of halogen atom, —OH, C₁-C₃-alkyloxy, —CONH₂ and phenyl;
a moiety selected from the group consisting of 3,4-dihydroquinolin-2(1H)-on-yl, 1,4-benzodioxanyl and benzofuranyl, which moiety is linked through carbon atom of its benzene ring; or
imidazolidin-2-on-yl linked through its nitrogen atom;
   and pharmaceutically acceptable salts thereof.

One group of compounds of the present invention are compounds of formula (IA), wherein A¹ represents phenyl unsubstituted or substituted with one substituent selected from the group consisting of halogen atom, —OH, C₁-C₃-alkyloxy, and phenyl; a moiety selected from the group consisting of 3,4-dihydroquinolin-2(1H)-on-yl, 1,4-benzodioxanyl and benzofuranyl, which moiety is linked through carbon atom of its benzene ring; or imidazolidin-2-on-yl linked through its nitrogen atom, and R¹, n, m have the meanings as defined above for formula (IA).

Another group of compounds of the present invention are compounds of formula (IA), wherein A¹ represents phenyl unsubstituted or substituted with one substituent selected from the group consisting of halogen atom, —OH, C₁-C₃-alkyloxy, —CONH₂ and phenyl; a moiety selected from the group consisting of 3,4-dihydroquinolin-2(1H)-on-yl, 1,4-benzodioxanyl and benzofuranyl, which moiety is linked through carbon atom of its benzene ring; and R¹, n, m have the meanings as defined above for formula (IA).

Further group of compounds of the present invention are compounds of formula (IA), wherein A¹ represents 3,4-dihydroquinolin-2(1H)-on-yl. Preferably, in this group A¹ represents 3,4-dihydroquinolin-2(1H)-on-7-yl.

Another group of compounds of the present invention are compounds of formula (IA), wherein A¹ represents unsubstituted phenyl.

Yet another group of compounds of the present invention are compounds of formula (IA), wherein A¹ represents phenyl substituted with one substituent selected from the group consisting of halogen atom, —OH, C₁-C₃-alkyloxy, —CONH₂ and phenyl. Preferably, the substituent is selected from the group consisting of halogen atom, C₁-C₃-alkyloxy and —CONH₂.

Further group of the compounds of the present invention are compounds of formula (IA), wherein R¹ represents unsubstituted phenylsulphonyl.

Yet further group of the compounds of the present invention are compounds of formula (IA), wherein R¹ represents unsubstituted benzyl.

Another group of compounds of the invention are compounds of formula (IA), wherein R¹ represents benzyl substituted with halogen atom, preferably with fluorine or chlorine.

A preferred sub-group of compounds of the invention are compounds of formula (IA), wherein A¹ represents 3,4-dihydroquinolin-2(1H)-on-yl, R¹ represents unsubstituted phenylsulphonyl.

Further preferred sub-group of compounds of the invention are compounds of formula (IA), wherein A¹ represents 3,4-dihydroquinolin-2(1H)-on-yl, and R¹ represents unsubstituted benzyl.

Yet further sub-group of compounds of the invention are compounds of formula (IA), wherein A¹ represents 3,4-dihydroquinolin-2(1H)-on-yl, and R¹ represents benzyl substituted with halogen atom, preferably with fluorine or chlorine.

A preferred sub-group of compounds of the invention are compounds of formula (IA), wherein A¹ represents unsubstituted phenyl and R¹ represents unsubstituted phenylsulphonyl.

Another sub-group of compounds of the invention are compounds of formula (IA), wherein A¹ represents unsubstituted phenyl and R¹ represents unsubstituted benzyl.

Yet further sub-group of compounds of the invention are compounds of formula (IA), wherein A¹ represents unsubstituted phenyl and R¹ represents benzyl substituted with halogen atom, preferably with fluorine or chlorine.

A preferred sub-group of compounds of the invention are compounds of formula (IA), wherein A¹ represents phenyl substituted with one substituent selected from the group consisting of halogen atom, —OH, C₁-C₃-alkyloxy, —CONH₂ and phenyl, preferably of halogen atom, C₁-C₃-alkyloxy and —CONH₂, and R¹ represents unsubstituted phenylsulphonyl.

Another sub-group of compounds of the invention are compounds of formula (IA), wherein A¹ represents phenyl substituted with one substituent selected from the group consisting of halogen atom, —OH, C₁-C₃-alkyloxy, —CONH₂ and phenyl, preferably of halogen atom, C₁-C₃-alkyloxy and —CONH₂, and R¹ represents unsubstituted benzyl.

Yet further sub-group of compounds of the invention are compounds of formula (IA), wherein A¹ represents phenyl substituted with one substituent selected from the group consisting of halogen atom, —OH, C₁-C₃-alkyloxy, —CONH₂ and phenyl, preferably of halogen atom. C₁-C₃-alkyloxy and —CONH₂, and R¹ represents benzyl substituted with halogen atom, preferably with fluorine or chlorine.

The following specific compounds of formula (IA) of the invention can be mentioned:
7-[3-[4-(1-benzylindol-4-yl)piperazin-1-yl]propoxy]-3,4-dihydro-1H-quinolin-2-one
7-(4-(4-(1-benzyl-1H-indol-4-yl)piperazin-1-yl)butoxy)-3,4-dihydroquinolin-2 (1H)-one
7-[3-[4-[1-[(2-fluorophenyl)methyl]indol-4-yl]piperazin-1-yl]propoxy]-3,4-dihydro-1H-quinolin-2-one
7-[3-[4-[1-[(3-fluorophenyl)methyl]indol-4-yl]piperazin-1-yl]propoxy]-3,4-dihydro-1H-quinolin-2-one
7-[3-[4-[1[(3-hydroxyphenyl)methyl]indol-4-iyl]piperazin-1-yl]propoxy]-3,4-dihydro-1H-quinolin-2-one
7-[3-[4-[1-(m-tolylmethyl)indol-4-yl]piperazin-1-yl]propoxy]-3,4-dihydro-1H-quinolin-2-one
4-(4-(3-phenoxypropyl)piperazin-1-yl)-1-(phenylsulphonyl)-1H-indole
4-(4-(3-(4-fluorophenoxy)propyl)piperazin-1-yl)-1-(phenylsulphonyl)-1H-indole
4-(4-(3-(2-(1-methylethoxy)phenoxy)propyl)piperazin-1-yl)-1-(phenyl-sulphonyl)-1H-indole
7-[3-[4-[1-(benzensulphonyl)indol-4-yl]piperazin-1-yl]propoxy]-3,4-dihydro-1H-quinolin-2-one
7-(4-(4-(1-(phenylsulphonyl)-1H-indol-4-yl)piperazin-1-yl)butoxy)-3,4-dihydroquinolin-2(1H)-one
4-(4-(3-(benzofuran-6-yloxy)propyl)piperazin-1-yl)-1-(phenylsulphonyl)-1H-indole
.2-[3-[4-(1-benzylindol-4-yl)piperazin-1-yl]propoxy]benzamide
7-[4-[4-[1-[(2-fluorophenyl)methyl]indol-4-yl]piperazin-1-yl]butoxy]-3,4-dihydro-1H-quinolin-2-one
2-[3-[4-[1-[(2-fluorophenyl)methyl]indol-4-yl]piperazin-1-yl]propoxy]benzamide
7-[4-[4-[1-[(3-fluorophenyl)methyl]indol-4-yl]piperazin-1-yl]butoxy]-3,4-dihydro-1H-quinolin-2-one
2-[3-[4-[1-[(3-fluorophenyl)methyl]indol-4-yl]piperazin-1-yl]propoxy]benzamide
7-[4-[4-[1-[(4-fluorophenyl)methyl]indol-4-yl]piperazin-1-yl]butoxy]-3,4-dihydro-1H-quinolin-2-one
2-[3-[4-[1-[(4-fluorophenyl)methyl]indol-4-yl]piperazin-1-yl]propoxy]-benzamide
7-[4-[4-[1-[(3-chlorophenyl)methyl]indol-4-yl]piperazin-1-yl]butoxy]-3,4-dihydro-1H-quinolin-2-one
2-[3-[4-[1-[(3-chlorophenyl)methyl]indol-4-yl]piperazin-1-yl]propoxy]-benzamide
1-(benzenesulfonyl)-4-[4-(4-phenoxybutyl)piperazin-1-yl]indole
1-(benzenesulfonyl)-4-[4-[4-(4-fluorophenoxy)butyl]piperazin-1-yl]-indole
1-(benzenesulfonyl)-4-[4-[4-(2-isopropoxyphenoxy)butyl]-piperazin-1-yl]-indole
2-[3-[4-[1-(benzenesulfonyl)indol-4-yl]piperazin-1-yl]propoxy]-benzamide
2-[4-[4-[1-(benzenesulfonyl)indol-4-yl]piperazin-1-yl]butoxy]-benzamide
   and pharmaceutically acceptable salts and solvates thereof.

Indoleamine derivatives of the above formula (IA) exhibit affinity to receptors which are recognized therapeutical targets in the treatment of CNS disorders, such as to dopaminergic, in particular D2 and D3, serotoninergic, in particular 5-HT1A, 5-HT2A, 5-HT6,5-HT7, and adrenergic, in particular α1 and α2C. Moreover, compounds of formula (IA) reveal affinity for serotonine transporter (SERT, 5-HTT) and have low affinity toward biological targets associated with adverse effects, such as potassium channel hERG, muscarinic receptors M3, histaminergic receptors H1 or serotoninergic receptors is 5-HT2C. Due to such a broad pharmacological profile, the compounds of the invention may be useful in medicine as medicaments, for the treatment and/or prevention of the central nervous system disorders such as schizophrenia, schizoaffective disorders, schizophreniform disorders, delusional syndromes and other psychotic conditions related and not related to taking psychoactive substances, depression, affective bipolar so disorder, mania and depression episodes, anxiety disorders of various etiology, consciousness disorders including coma, delirium of alcoholic or other etiology, aggression, psychomotor agitation and other conduct disorders, sleep disorders of various etiology, withdrawal syndromes of various etiology, addiction, pain syndromes of various etiology, intoxication with psychoactive substances, cerebral circulatory disorders of various etiology, psychosomatic disorders of various etiology, conversion disorders, dissociative disorders, urination disorders, autism and other developmental disorders, including nocturia, stuttering, tics, cognitive disorders of various types, such as Alzheimer's disease, psychopathological symptoms and neurological disorders in the course of other diseases of the central and peripheral nervous systems.

Thus, the subject of the present invention are the compounds of formula (IA) as defined above, for use as a medicament.

In the treatment of central nervous system disorders compounds of formula (IA) may be administered in the form of a pharmaceutical composition or preparation containing it. Thus, the subject of the present invention is also the pharmaceutical composition containing the compound or compounds of formula (IA) as defined above as an active substance, in combination with pharmaceutically acceptable carrier(s) and/or excipient(s).

The subject of the invention is also a use of indole derivatives of the above formula (IA) for the treatment of disorders of central nervous system.

The invention relates also to a method for the treatment of disorders of the central nervous system in mammals, including humans, comprising administration of a therapeutically effective amount of the compound of above formula (IA) or the pharmaceutical composition containing the compound of formula (IA) as defined above as an active substance.

Terms used in the description of the present invention have the following meanings.

The term “C₁-C₃-alkyl” relates to a saturated, straight or branched hydrocarbon group, having indicated number of carbon atoms. Specific examples of groups encompassed by this term are methyl, ethyl, n-propyl, isopropyl.

The term “halogen atom” relates to a substituent selected from F, Cl, Br and I.

The term “C₁-C₃-alkyloxy” relates to —O—C₁-C₃-alkyl group, wherein C₁-C₃-alkyl relates to a saturated, straight or branched hydrocarbon group, having indicated number of carbon atoms. Specific examples of groups encompassed by this term are methoxy, ethoxy, n-propoxy, isopropoxy.

The compounds of formula (IA) according to the invention can be prepared in a process presented in the following scheme:

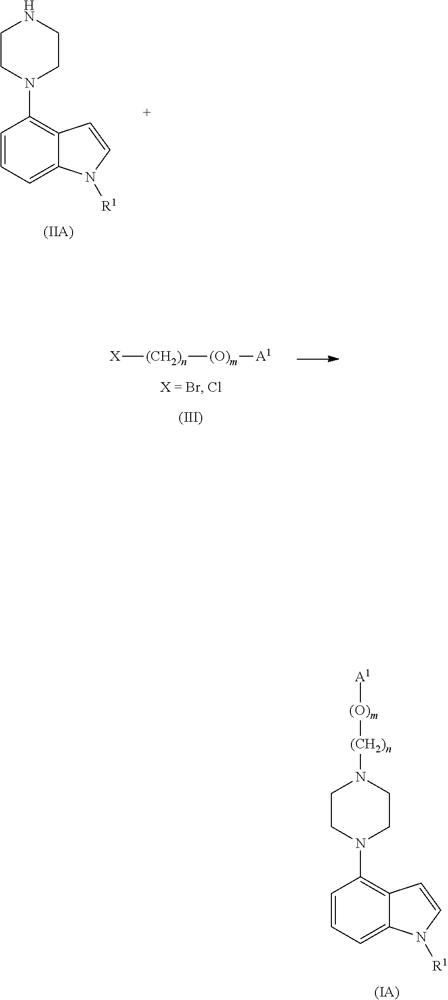

The appropriate secondary amine of formula A acid addition salt thereof, preferably hydrochloride salt, is subjected to reaction N-alkylation with the appropriate halogen derivative of formula (III) in the presence of the excess of a base, for example triethylamine or potassium carbonate, optionally in the presence of a catalytic amount of potassium iodide at elevated temperature to give a compound of formula (IA) of the invention. Reaction is carried out for example at 70° C. in acetonitrile. Reaction time is usually from 8 to 12 hours.

Starting secondary amines of formula (IIA) can be prepared by the methods known in the art. Amine of formula (IIA) as hydrochloride can be obtained by the method presented on the following scheme:

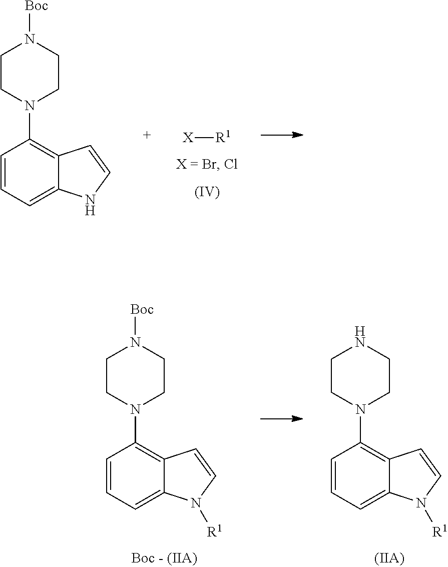

Commercially available 4-(4-Boc-piperazin-1-yl)-1H-indole is subjected to nucleophilic substitution reaction with an appropriate halogen derivative of formula (IV) in the is presence of a base, for example, potassium tert-butoxide and in the presence of a crown ether catalyst. Reaction is carried out initially at low temperature, for example at 0° C., and then continued at ambient temperature in anhydrous tetrahydrofuran as the solvent. Reaction time is usually from 10 to 14 hours. The product of substitution reaction, amine Boc-(IIA), is deprotected using 4M hydrogen chloride solution in dioxane and the resulting amine of formula (IIA) as the hydrochloride salt is used without further purification in the next step of synthesis of compounds of formula (IA) of the invention.

Halogen derivatives of formula (IV) are known and commercially available.

Halogen derivatives of formula (III) are either well known or commercially available, or can be prepared from commercially available starting materials by adapting and applying known methods.

Preparation of exemplary compounds of formula (IA) and starting compounds of formula (IIA) is described in detail in the experimental part.

Since the compounds of formula (IA) have alkaline character (contain at least one tertiary amine group), they can form acid addition salts.

Salts with acids can be pharmaceutically acceptable, especially when they are intended to be an active ingredient in pharmaceutical composition. The present invention relates also to salts of the compounds of formula (IA) with acids other than pharmaceutically acceptable ones, which may be useful for example as intermediates suitable for purification of the compounds of the invention. In practice, it is often desirable to isolate first the compound from a reaction mixture in the form of a salt which is not pharmaceutically acceptable to purify the compound, and then convert the salt into is free base by treatment with alkaline agent and to isolate, and optionally convert into the salt again.

Acid addition salts can be formed with inorganic (mineral) or organic acids. In particular, hydrochloric, hydrobromic, hydroiodic, phosphoric, sulphuric, nitric, carbonic, succinic, maleic, formic, acetic, propionic, fumaric, citric, tartaric, lactic, benzoic, salicylic, glutamic, aspargic, p-toluenesulphonic, benzenesulphonic, methanesulphonic, ethanesulphonic, naphthalenesulphonic such as 2-naphthalene-sulphonic, pamoic, xinafoic or hexanoic acids can be mentioned as examples of acids.

Acid addition salt can be prepared in a simple manner by reaction of the compound of formula (IA) with suitable inorganic or organic acid, optionally in suitable solvent, such as organic solvent, to form a salt that is usually isolated, for example by crystallization and filtration. For example, compounds in the form of a free base can be converted into corresponding hydrochloride salts by reaction of a compound in a solution, for example in methanol, with stoichiometric amount of hydrochloric acid or with solution of hydrochloric acid in methanol, ethanol or diethyl ether, followed by evaporation of solvent(s).

The term “disorders of the central nervous system” should be understood as including disorders selected from schizophrenia, schizoaffective disorders, schizophreniform disorders, delusional syndromes and other psychotic conditions related and not related to taking psychoactive substances, affective disorder, bipolar disorder, mania, depression, anxiety disorders of various etiology, stress reactions, consciousness disorders, coma, delirium of alcoholic and other etiology, aggression, psychomotor agitation and other conduct disorders, sleep disorders of various etiology, withdrawal syndromes of various etiology, addiction, pain syndromes of various etiology, intoxication with psychoactive substances, cerebral circulatory disorders of various etiology, psychosomatic disorders of various etiology, conversion disorders, dissociative disorders, urination disorders, autism and other developmental disorders, including nocturia, stuttering, and tics, cognitive disorders of various types, like Alzheimer's disease, psychopathological symptoms and neurological disorders in the course of other diseases of the central and peripheral nervous systems.

In the treatment of the disorders mentioned above, compounds of formula (IA) of the present invention can be administered as a chemical compound, but usually will be applied in the form of a pharmaceutical compositions containing the compound of the present invention or its pharmaceutically acceptable salt as defined above as an active ingredient in combination with pharmaceutically acceptable carrier(s) and/or is excipient(s).

In the treatment of the above mentioned disorders the pharmaceutical compositions of the invention can be delivered by any route of administration, preferably oral or parenteral, and will have the form of a preparation for use in medicine, depending on the intended route of administration.

Compositions for oral administration may have the form of solid or liquid preparations. Solid preparations may be in the form, for example, tablets or capsules prepared in conventional manner using pharmaceutically acceptable inactive ingredients, such as binding agents (e.g. pregelatinized maize starch, polyvinylpyrrolidone or hydroxypropyl-methylcellulose); fillers (e.g. lactose, sucrose, carboxymethylcellulose, microcrystalline cellulose or calcium hydrogen phosphate) lubricants (e.g. magnesium stearate, talc or silica); disintegrants (e.g. crospovidone, maize starch or sodium starch glycolate); wetting agents (e.g. sodium lauryl sulfate). The tablets may be coated using methods well known in the art with conventional coatings, delaying/controlling release coatings or enteric coatings. Liquid preparations for oral administration may have the form of e.g. solutions, syrups or suspensions, or may be prepared from a dry product suitable for reconstitution with water or other suitable carrier ex tempore. Such liquid preparations may be prepared by conventional methods with pharmaceutically acceptable inactive ingredients, such as suspending agents (e.g. sorbitol syrup, cellulose derivatives or hydrogenated edible fats), emulsifying agents (e.g. lecithin or acacia gum), non-aqueous matrix components (e.g. almond oil, oils esters, ethyl alcohol or fractionated vegetable oils) and preservatives (e.g. methyl or propyl p-hydroxybenzoates or sorbic acid). The preparations may also contain suitable buffering systems, flavouring and aroma agents, colourants and sweeteners.

Preparations for oral administration can be formulated according to methods well known to those skilled in the art to afford a controlled release of the active compound.

The parenteral route of administration comprises administration by intramuscular and intravenous injections and intravenous continuous infusions. Compositions for parenteral administration may be in the form of a dosage unit, e.g. in ampoules or in multidose containers with the addition of a preservative. The compositions may be in the form of suspensions, solutions or emulsions in oily or aqueous media, and may contain pharmaceutically acceptable excipients, such as suspending agents, stabilizing and/or dispersing agents. Alternatively, the active ingredient may be in the form of a powder for reconstitution ex tempore in a suitable carrier, e.g. sterile pyrogen-free water.

Method of treatment using compounds of this invention will be based on administration of a therapeutically effective amount of the compound of the invention, preferably in the form of a pharmaceutical composition, to a subject in need of such a treatment.

The proposed dose of the compounds of the invention will be comprised in the range from 1 to about 1000 mg per day, in a single dose or in divided doses. It will be apparent to those skilled in the art that selection of a dose required to achieve the desired biological effect will depend on several factors, such as the type of specific compound, the indication, route of administration, age and condition of a patient and the exact dose will be finally determined at the discretion of attending physician.

### EXAMPLE 1

### Preparation of Starting Amines of Formula (IIA) and Halogen Derivatives of Formula (III)

### General Procedure for Preparation of Amines of Formula (IIA)

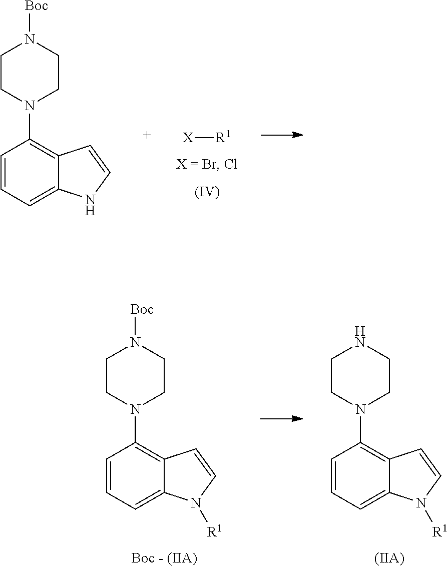

To a solution of potassium tert-butoxide (2.16 ml of 1M solution in tetrahydrofuran, 1.3 eq.) in 7 ml of dry tetrahydrofuran and crown ether 18-crown-6 (0.2 eq.) a solution of 4-tert-butyl (1H-indol-4-yl)-piperazine-1-carboxylate (Boc-P) (1.66 mmol, 1 eq.) in 10 ml of dry tetrahydrofuran was added dropwise at 0° C. After 20 minutes halogen derivative (IV) (2.49 mmol, 1.5 eq.) was added and the resulting suspension was stirred overnight at ambient temperature. Then, tetrahydrofuran was evaporated under reduced pressure and to the residue ethyl acetate was added and the mixture was extracted with water. The organic layer was dried over anhydrous sodium sulfate, and after evaporation of the solvent the crude reaction mixture was purified by column chromatography using the solvent system hexane/ethyl acetate 8:2, yielding a solid product Boc-(IIA). Yield of Boc-(IIA) was in the range of 85-95%.

Compounds Boc-(IIA) were deprotected according to the following procedure.

A mixture of amine Boc-(IIA) (0.589 mmol) and 4M hydrogen chloride solution in dioxane (3 ml) was stirred at room temperature for 20 minutes, and then the solvent was to evaporated under reduced pressure. Deprotected amine (IIA) as the hydrochloride salt was obtained in a yield of about 95% and was used without further purification in the next steps of synthesis of compounds of formula (IA) of the invention.

According to the above procedure the following amines of formula (IIA) were prepared:
1-benzyl-4-piperazin-1-yl-1H-indole (IIA-1) as hydrochloride was prepared from Boc-P and benzyl bromide, MS: 292 [M+H⁺],
1-(2-fluorobenzyl)-4-piperazin-1-yl-1H-indole (IIA-2) as hydrochloride was prepared from Boc-P and 2-fluorobenzyl bromide, MS: 310 [M+H⁺],
1-(3-fluorobenzyl)-4-piperazin-1-yl-1H-indole (IIA-3) as hydrochloride was prepared from Boc-P and 3-fluorobenzyl bromide, MS: 310 [M+H⁺],
3[(4-piperazin-1-yl-1H-indol-1-yl)methyl]phenol (IIA-4) as hydrochloride was prepared from Boc-P and 3-(bromomethyl)phenol, MS: 308 [M+H⁺],
1-(3-methylbenzyl)-4-piperazin-1-yl-1H-indole (IIA-5) as hydrochloride was prepared from Boc-P and 1-(bromomethyl)-3-methylbenzene, MS: 306 [M+H⁺],
1-(phenylsulphonyl)-4-piperazin-1-yl-1H-indole (IIA-6) as hydrochloride was prepared from Boc-P and benzenesulphonyl chloride, MS: 342 [M+H⁺],
1-[(2-fluorophenyl)sulphonyl]-4-piperazin-1-yl-1H-indole (IIA-7) as hydrochloride was prepared from Boc-P and 2-fluorobenzenesulphonyl chloride, MS: 360 [M+H⁺],
1-[(3-fluorophenyl)sulphonyl]-4-piperazin-1-yl-1H-indole (IIA-8) as hydrochloride was prepared from Boc-P and 3-fluorobenzenesulphonyl chloride, MS: 360 [M+H⁺],
1-[(4-fluorophenyl)sulphonyl]-4-piperazin-1-yl-1H-indole (IIA-9) as hydrochloride was prepared from Boc-P and 4-fluorobenzenesulphonyl chloride, MS: 360 [M+H⁺],
3-[(4-piperazin-1-yl-1H-indol-1-yl)sulphonyl]phenol (IIA-10) as hydrochloride was prepared from Boc-P and 3-hydroxybenzenesulphonyl chloride, MS: 358 [M+H⁺],
1-[(3-methylphenyl)sulphonyl]-4-piperazin-1-yl-1H-indole (IIA-11) as hydrochloride was prepared from Boc-P and 3-methylobenzenesulphonyl chloride, MS: 356 [M+H⁺],
1-(4-fluorobenzyl)-4-piperazin-1-yl-1H-indole IIA-16) as hydrochloride was prepared from Boc-P and 4-fluorobenzyl bromide, MS: 310 [M+H⁺],
1-(3-chlorobenzyl)-4-piperazin-1-yl-1H-indole (IIA-17) as hydrochloride was prepared from Boc-P and 3-chlorobenzyl bromide, MS: 326 [M+H⁺],
   Halogen derivatives (III) are either commercially available (when marked with asterisk*), or known and obtainable by methods described in literature:
(3-bromopropoxy)benzene (III-2)*,
1-(3-bromopropoxy)-4-fluorobenzene (III-5)*,
1-(3-chloropropoxy)-2-(1-methylethoxy)benzene (III-11) was prepared according to the method described in Walsh, David A. et al., Journal of Medicinal Chemistry, 32(1), 105-18; 1989,
7-(3-bromopropoxy)-3,4-dihydroquinolin-2(1H)-one (III-20) was prepared according to the method described in Banno, Kazuo et al., Chemical a Pharmaceutical Bulletin, 1988, 36(11), 4377-88,
7-(4-bromobutoxy)-3,4-dihydroquinolin-2(1H)-one (III-21) was prepared according to the method described in US2008/0293736,
6-(3-chloropropoxy)-1H-1-indole (III-22)*
6-(3-chloropropoxy)-1-benzofuran (III-23)*
(4-bromobutoxy)benzene (111-27)*,
1-(4-bromobutoxy)-4-fluorobenzene (III-28)*
1-(4-chlorobutoxy)-2-(1-methylethoxy)benzene (III-29) was prepared according to the method described in Walsh, David A. et al., Journal of Medicinal Chemistry, 32(1), 105-18; 1989,
2-(3-chloropropoxy)benzamide (III-30) was prepared according to the method described in Kowalski, P., J.Heterocyclic Chem., 48, 192-198, 2011,
2-(4-chlorobutoxy)benzamide (III-31) was prepared according to the method described in Kowalski, P., J.Heterocyclic Chem., 48, 192-198, 2011.

### EXAMPLE 2

### Procedures for the Preparation of Compounds (IA) According to the Invention

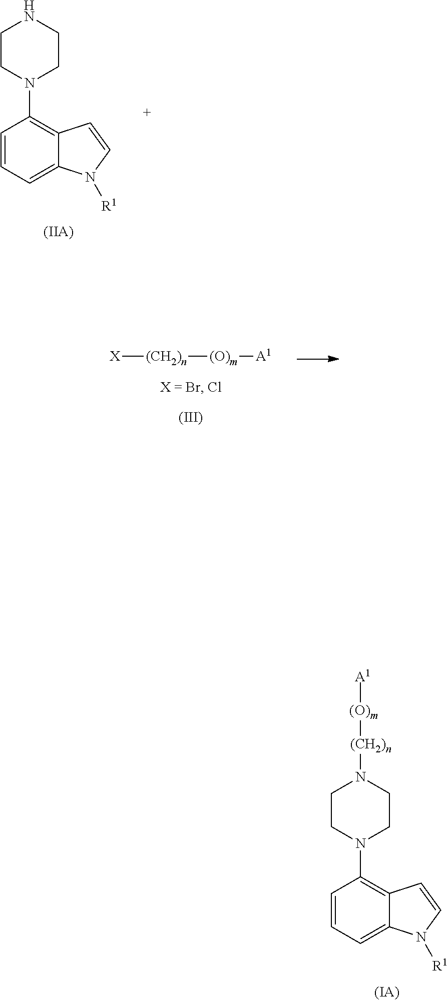

a1) A mixture of amine (IIA) hydrochloride (0.354 mmol, 1.0 eq.), halogen derivative of formula (III) (0.425 mmol, 1.2 eq.), triethylamine (0.788 mmol, 2.2 eq.) and potassium iodide (0.2 eq.) in 7 ml of acetonitrile was stirred for 8 hours at 70° C. Then, solvent was evaporated and the residue was purified by column chromatography in the solvent system methylene chloride/methanol 95:5 v/v, to obtain a compound of formula (IA) according to the invention with a yield in a range of 70-90%.

a2) A mixture of amine (IIA) hydrochloride (0.520 mmol, 1.2 eq.), halogen derivative of formula (III) (0.430 mmol, 1.0 eq.), potassium carbonate (1.29 mmol, 3 eq.) and potassium iodide (0.2 eq.) in 10 ml of acetonitrile was stirred for 12 h at 70° C. Then the inorganic precipitate was filtered off, solvent was evaporated from the filtrate and the residue was purified by column chromatography in the solvent system methylene chloride/methanol 95:5 v/v, to afford a compound of formula (IA) according to the invention with a yield in the range of 70-90%.

According to one of the above procedures a1 and a2, the following compounds of formula (IA) according to the invention were prepared.

### Compound 4. 7-[3-[4-(1-Benzylindol-4-yl)piperazin-1-yl]propoxy]-3,4-dihydro-1H-quinolin-2-one

The title compound was prepared starting from amine (IIA-1) and halogen derivative (III-20) according to the procedure a1).

¹H-NMR (300 MHz, CDCl₃): δ 8.12 (s, 1H), 7.34-7.26 (m, 2H), 6.96 (d, 1H, J=8.2 Hz), 7.16-7.08 (m, 5H), 6.63-6.50 (m, 4H), 6.35 (d, 1H, J=2.5 Hz), 5.3 (s, 2H), 3.80-3.62 (m, 6H), 3.18-3.04 (m, 4H), 2.82-2.74 (m, 4H), 2.38-2.30 (m, 2H), MS: 495 [M+H⁺].

### Compound 5. 7-(4-(4-(1-Benzyl-1H-indol-4-yl)piperazin-1-yl)butoxy)-3,4-dihydroquinolin-2(1H)-one

The title compound was prepared starting from amine (IIA-1) and halogen derivative (III-21) according to the procedure a1).

¹H-NMR (300 MHz, CDCl₃): δ 8.21 (s, 1H), 7.30-7.22 (m, 2H), 6.94 (d, 1H, J=8.2 Hz), 7.12-7.02 (m, 5H), 6.62-6.50 (m, 4H), 6.34 (d, 1H, J=2.5 Hz), 5.3 (5, 2H), 3.89 (t, 2H, J=6.4 Hz), 2.92-2.86 (m, 2H), 3.34-3.26 (m, 4H), 2.78-2.50 (m, 4H), 2.64-2.58 (m, 2H), 2.50 (t, 2H, J=7.4 Hz), 1.88-1.70 (m, 4H). MS: 509 [M+H⁺].

### Compound 6. 7-[3-[4-[1[(2-fluorophenyl)methyl]indol-4-yl]piperazin-1-yl]propoxy]-3,4-dihydro-1H-quinolin-2-one

The title compound was prepared starting from amine (IIA-2) and halogen derivative (III-20) according to the procedure a1). MS: 513 [M+H⁺]

### Compound 8. 7-[3-[4-[1-[(3-Fluorophenyl)methyl]indol-4-yl]piperazin-1 yl]propoxy]-3,4-dihydro-1H-quinolin-2-one

The title compound was prepared starting from amine (IIA-3) and halogen derivative (III-20) according to the procedure a1). MS: 513 [M+H⁺]

### Compound 9. 7-[3-[4-[1-[(3-Hydroxyphenyl)methyl]indol-4-iyl]piperazin-1-yl]propoxy]-3,4-dihydro-1H-quinolin-2-one

The title compound was prepared starting from amine (IIA-4) and halogen derivative (III-20) according to the procedure a1). MS: 511 [M+H⁺]

### Compound 10. 7-[3-[4-[1-(m-Tolylmethyl)indol-4-yl]piperazin-1-yl]propoxy]-3,4-dihydro-1H-quinolin-2-one

The title compound was prepared starting from amine (IIA-5) and halogen derivative (III-20) according to the procedure a1). MS: 509 [M+H⁺]

### Compound 12. 4-(4-(3-Phenoxypropyl)piperazin-1-yl)-1-(phenylsulphonyl)-1H-indole

The title compound was prepared starting from amine (IIA-6) and halogen derivative (III-2) according to the procedure a2).

¹H-NMR (300 MHz, CDCl₃): δ 7.90-7.86 (m, 2H), 7.68 (d, 1H, J=8.4 Hz), 7.56-7.52 (m, 2H), 7.48-7.40 (m, 3H), 7.32-7.20 (m, 3H), 6.98-6.86 (m, H), 6.76 (d, 1H, J=7.4 Hz), 6.64 (dd, 1H, J=3.8 i 0.7 Hz), 4.08 (t, 2H, J=5.4 Hz), 3.42-3.36 (m, 4H), 3.06-2.98 (m, 4H), 2.92 (t, 2H, J=5.2 Hz), 2.30-2.20 (m, 2H). MS: 476 [M+H⁺].

### Compound 15. 4-(4-(3-(4-Fluorophenoxy)propyl)piperazin-1-yl)-1-(phenylsulphonyl)-1H-indole

The title compound was prepared starting from amine (IIA-6) and halogen derivative (III-5) according to the procedure a2).

¹H-NMR (300 MHz, DMSO): δ 0.98-7.92 (m, 2H), 7.76-7.72 (m, 2H), 7.66 (d, 1H, J=7.4 Hz), 7.60-7.52 (m, 3H), 7.21 (t, 1H, J=7.9 Hz), 7.10 (t, 1H, J=8.9 Hz), 6.96-6.90 (m, 2H), 6.84-6.80 (m, 1H) 6.72 (d, 1H, J=7.4 Hz), 4.00 (t, 2H, J=6.1 Hz), 3.70-3.64 (m, 2H), 3.31-3.00 (m, 4H), 2.72-2.50 (m, 4H), 2.02-1.88 (m, 2H). MS: 494 [M+H⁺].

### Compound 21. 4-(4-(3-(2-(1-Methylethoxy)phenoxy)propyl)piperazin-1-0)-1-(phenylsulphonyl)-1H-indole

The title compound was prepared starting from amine (IIA-6) and halogen derivative (III-11) according to the procedure a2).

¹H-NMR (300 MHz, CDCl₃): δ 7.90-7.84 (m, 3H), 7.66 (d, 1H, J=8.2 Hz), 7.54-7.50 (m, 2H), 7.48-7.40 (m, 2H), 7.22 (t, 1H, J=7.9 Hz), 6.68 (d, 1H, J=3.8 Hz), 4.08 (t, 2H, J=5.4 Hz), 3.45-3.38 (m, 4H), 2.60-2.40 (m, 4H), 2.10-2.00 (m, 2H), 1.45 (d, 6H, J=6.1 Hz). MS: 534 [M+H⁺].

### Compound 23. 7-[3-[4-[1-(Benzensulphonyl)indol-4-yl]piperazin-1-yl]propoxy]-3,4-dihydro-1H-quinolin-2-one

The title compound was prepared starting from amine (IIA-1) and halogen derivative (III-20) according to the procedure a1).

¹H-NMR (300 MHz, DMSO): δ 7.95 (d, 2H, J=7.4 Hz), 7.80 (d, 1H, J=3.8 Hz), 7.60-7.51 (m, 3H), 7.35 (t, 2H, J=8.2 Hz), 7.02 (d, 1H, J=8.2 Hz), 6.80 (d, 1H, J=3.6 Hz), 6.75 (d, 1H, J=7.7 Hz), 6.50-6.40 (m, 2H), 4.00 (m, 2H), 3.56-3.46 (m, 6H), 3.12-3.00 (m, 4H), 2.80-2.70 (m, 4H), 2.40-2.20 (m, 2H). MS: 545 [M+H⁺].

### Compound 24. 7-(4-(4-(1-(Phenylsulphonyl)-1H-indol-4-yl)piperazin-1-yl)butoxy)-3,4-dihydroquinolin-2(1H)-one

The title compound was prepared starting from amine (IIA-6) and halogen derivative (III-21) according to the procedure a1).

¹H-NMR (300 MHz, CDCl₃): δ 8.26 (s, 1H), 7.90-7.82 (m, 2H), 7.64 (d, 1H, J=8.4 Hz), 7.54-7.49 (m, 2H), 7.44-7.38 (m, 2H), 7.22 (t, 1H, J=8.2 Hz), 7.02 (d, 1H, J=8.4 Hz), 6.72 (d, 1H, J=7.6 Hz), 6.68 (d, 1H, J=3.8 Hz), 6.50 (dd, 2H, J=8.2 i 2.3 Hz), 6.32 (d, 1H, J=2.5 Hz), 3.74 (t, 2H, J=6.3 Hz), 3.20-3.12 (m, 4H), 2.88 (t, 2H, J=7.5 Hz), 2.50-2.36 (m, 6H), 2.48 (t, 2H, J=7.4 Hz), 1.90-1.64 (m, 4H). MS: 559 [M+H⁺].

### Compound 25. 4-(4-(3-(Benzofuran-6-yloxy)propyl)piperazin-1-yl)-1-(phenylsulphonyl)-1H-indole

The title compound was prepared starting from amine (IIA-6) and halogen derivative (III-23) according to the procedure a2).

¹H-NMR (300 MHz, CDCl₃): δ 788-7.84 (m, 2H), 7.64 (d, 1H, J=7.9 Hz), 7.54-7.38 (m, 5H), 7.22 (t, 2H, J=8.2 Hz), 7.04 (d, 1H, J=7.4 Hz), 6.88 (dd, 1H, J=8.4 i 2.0 Hz), 6.72 (d, 1H, J=7.4 Hz), 6.70-6.68 (m, 2H), 4.10 (t, 2H, J=6.1 Hz), 3.22-3.05 (m, 5H), 2.80-2.60 (m, 5H), 2.10-2.00 (m, 2H). MS: 516 [M+H⁺].

### Compound 60. 2-[3-[4-(1-Benzylindol-4-yl)piperazin-1-yl]propoxy]benzamide

The title compound was prepared starting from amine (IIA-1) and halogen derivative (III-30) according to the procedure a1).

¹H-NMR (300 MHz, CDCl₃): δ 8.21 (dd, 2H, J=1.8 and 7.9 Hz), 7.91-7.88 (m, 1H), 7.50-7.42 (m, 1H), 7.30-7.22 (m, 3H), 7.10-6.94 (m, 6H), 6.60 (d, 1H, J=6.9 Hz), 6.52 (dd, 1H, J=0.7 and 3.0 Hz), 5.90-5.85 (m, 1H), 5.23 (s, 2H), 4.25 (t, 2H, J=6.4 Hz), 3.3-3.28 (m, 4H), 2.77-2.70 (m, 4H), 2.63 (t, 2H, J=7.1 Hz), 2.18-2.10 (m, 2H). MS: 469 [M+H⁺].

### Compound 61. 7-[4-[4-[1-[(2-Fluorophenyl)methyl]indol-4-yl]piperazin-1-yl]butoxy]-3,4-dihydro-1H-quinolin-2-one

The title compound was prepared starting from amine (IIA-2) and halogen derivative (III-21) according to the procedure a1).

¹H-NMR (300 MHz, CDCl₃): δ 7.50 (s, 1H), 7.23-7.20 (m, 1H), 7.13-6.92 (m, 6H), 6.84-6.78 (m, 1H), 6.62-6.50 (m, 2H), 6.28 (d, 1H, J=2.3 Hz), 5.34 (s, 2H), 3.98 (m, 2H, J=6.1 Hz), 3.62-3.58 (m, 4H), 2.79 (t, 2H, J=6.9 Hz), 2.78-2.70 (m, 4H), 2.60-2.50 (m, 4H), 1.90-1.70 (m, 2H), 1.70-1.60 (m, 2H). MS: 527 [M+H⁺].

### Compound 62. 2-[3-[4-[1-[(2-Fluorophenyl)methyl]indol-4-yl]piperazin-1-yl]propoxy]-benzamide

The title compound was prepared starting from amine (IIA-2) and halogen derivative (III-30) according to the procedure a1).

¹H-NMR (300 MHz, CDCl₃): δ 8.21 (dd, 2H, J=1.8 and 7.7 Hz), 7.91-7.88 (m, 1H), 7.50-7.42 (m, 1H), 7.14-6.94 (m, 7H), 6.85-6.78 (m, 1H), 6.60 (d, 1H, J=6.9 Hz), 6.54 (d, 1H, J=2.5 Hz), 5.80-5.76 (m, 1H), 5.28 (s, 2H), 4.25 (t, 2H, J=6.4 Hz), 3.35-3.25 (m, 4H), 2.80-2.70 (m, 4H), 2.70-2.60 (m, 2H), 2.18-2.10 (m, 2H). MS: 487 [M+H⁺].

### Compound 63. 7-[4-[4-[1-[(3-Fluorophenyl)methyl]indol-4-yl]piperazin-1-yl]butoxy]-3,4-dihydro-1H-quinolin-2-one

The title compound was prepared starting from amine (IIA-3) and halogen derivative (III-21) according to the procedure a1).

¹H-NMR (300 MHz, CDCl₃): δ 8.02 (s, 1H), 7.28-7.20 (m, 2H), 7.09-7.02 (m, 2H), 6.94-6.84 (m, 2H), 6.78-6.72 (m, 1H), 6.62-6.50 (m, 4H), 6.32 (d, 1H, J=2.3 Hz), 5.21 (s, 2H), 3.98 (t, 2H, J=6.1 Hz), 3.37-3.23 (m, 4H), 2.89 (m, 2H, J=6.9 Hz), 2.78-2.68 (m, 4H), 2.64-2.58 (m, 2H), 2.51 (t, 211, J=7.4 Hz) 1.89-1.69 (m, 4H). MS: 527 [M+H⁺].

### Compound 64. 2-[3-[4-[1-[(3-Fluorophenyl)methyl]indol-4-yl]piperazin-1-yl]propoxy]-benzamide

The title compound was prepared starting from amine (IIA-3) and halogen derivative (III-30) according to the procedure a1).

¹H-NMR (300 MHz, CDCl₃): δ 8.20 (dd, 2H, J=1.8 and 7.9 Hz), 7.49-7.42 (m, 1H), 7.28-7.20 (m, 2H), 7.13-7.04 (m, 3H), 7.03-6.85 (m, 4H), 6.78-6.72 (m, 1H), 6.61 (d, 1H, J=7.4 Hz), 6.52 (d, 1H, J=2.5 Hz), 5.25 (s, 2H), 4.30 (t, 2H, J=6.1 Hz), 3.40-3.32 (m, 4H), 2.92-2.82 (m, 4H), 2.82-2.73 (m, 2H), 2.30-2.20 (m, 2H). MS: 487 [M+H⁺].

### Compound 65. 7-[4-[4-[1-[(4-Fluorophenyl)methyl]indol-4-yl]piperazin-1-yl]butoxy]-3,4-dihydro-1H-quinolin-2-one

The title compound was prepared starting from amine (IIA-16) and halogen derivative (III-21) according to the procedure a1).

¹H-NMR (300 MHz, CDCl₃): δ 8.58 (s, 1H), 7.10-7.00 (m, 5H), 7.00-6.90 (m, 3H), 6.60 (d, 1H, J=7.4 Hz), 6.55-6.50 (m, 2H), 6.35 (d, 1H, J=2.3 Hz), 5.35 (s, 2H), 3.98 (t, 2H, J=6.1 Hz). 3.38-3.28 (m, 4H), 2.92-2.85 (t, 2H, J=6.9 Hz), 2.75-2.68 (m, 4H), 2.63-2.59 (m, 2H), 2.53-2.47 (t, 2H, J=7.4 Hz), 1.90-1.67 (m, 4H). MS: 527 [M+H⁺].

### Compound 66. 2-[3-[4-[1-[(4-Fluorophenyl)methyl]indol-4-yl]piperazin-1-yl]propoxy]-benzamide

The title compound was prepared starting from amine (IIA-16) and halogen derivative (III-30) according to the procedure a1).

¹H-NMR (300 MHz, CDCl₃): δ 8.22 (dd, 2H, J=1.8 and 7.9 Hz), 7.91-7.88 (m, 1H), 7.50-7.42 (m, 1H), 7.12-6.90 (m, 8H), 6.60 (d, 1H, J=6.9 Hz), 6.52 (dd, 1H, J=0.7 and 3.0 Hz), 5.88-5.80 (m, 1H), 5.25 (s, 2H), 4.25 (t, 2H, J=6.4 Hz), 3.31-3.25 (m, 4H), 2.80-2.70 (m, 4H), 2.62 (t, 2H, J=7.1 Hz), 2.28-2.20 (m, 2H). MS: 487 [M+H⁺].

### Compound 67. 7-[4-[4-[1-[(3-Chlorophenyl)methyl]indol-4-yl]piperazin-1-yl]butoxy]-3,4-dihydro-1H-quinolin-2-one

The title compound was prepared starting from amine (IIA-17) and halogen derivative (III-21) according to the procedure a1).

¹H-NMR (300 MHz, CDCl₃): δ 8.01 (s, 1H), 7.25-7.19 (m, 2H), 7.09-6.87 (m, 4H), 6.75-6.70 (m, 1H), 6.66-6.58 (m, 4H), 6.30 (d, 1H, J=2.3 Hz), 5.21 (s, 2H), 3.98 (t, 2H, J=6.1 Hz), 3.35-3.22 (m, 4H), 2.87 (m, 2H, J=6.9 Hz), 2.76-2.67 (m, 4H), 2.63-2.58 (m, 2H), 2.50 (t, 2H, J=7.4 Hz) 1.89-1.69 (m, 4H). MS: 544 [M+H⁺].

### Compound 68. 2-[3-[4-[1-[(3-Chlorophenyl)methyl]indol-4-yl]piperazin-1-yl]propoxy]-benzamide

The title compound was prepared starting from amine (IIA-17) and halogen derivative (III-30) according to the procedure a1).

¹H-NMR (300 MHz, CDCl₃): δ 7.91 (dd, 2H, J=1.8 and 7.9 Hz), 7.36-7.28 (m, 2H), 7.06-7.02 (m, 1H), 6.98-6.86 (m, 5H), 6.82-6.76 (m, 3H), 6.46 (d, 1H, J=7.4 Hz), 6.38 (dd, 1H, J=0.6 and 3.1 Hz), 5.18 (s, 2H), 4.10-4.05 (m, 2H), 3.20-3.15 (m, 4H), 2.74-2.68 to (m, 4H), 2.60 (t, 2H, J=7.2 Hz), 2.06-2.00 (m, 2H). MS: 503 [M+H⁺].

### Compound 69. 1(Benzenesulfonyl)-4-[4-(4-phenoxybutyl)piperazin-1-yl]indole

The title compound was prepared starting from amine (IIA-6) and halogen derivative (III-27) according to the procedure a2).

¹H-NMR (300 MHz, CDCl₃): 7.90-7.84 (m, 2H), 7.65 (d, 1H, J=8.4 Hz), 7.54-7.48 (m, 2H), 7.45-7.38 (m, 4H), 7.30-7.24 (m, 3H), 7.20 (t, 1H, J=7.9 Hz), 6.98-6.88 (m, 2H), 4.00 (t, 2H, J=6.4 Hz), 3.20-3.12 (m, 4H), 2.70-2.62 (m, 4H), 2.49 (t, 2H, J=7.6 Hz), 1.90-1.80 (m, 2H), 1.80-1.68 (m, 2H). MS: 490 [M+H⁺].

### Compound 70. 1-(Benzenesulfonyl)-4-[4-[4-(4-fluorophenoxy)butyl]piperazin-1-yl]-indole

The title compound was prepared starting from amine (IIA-6) and halogen derivative (III-28) according to the procedure a2).

¹H-NMR (300 MHz, CDCl₃): δ 7.89-7.84 (m, 2H), 7.64 (d, 1H, J=8.4 Hz), 7.55-7.48 (m, 2H), 7.45-7.38 (m, 2H), 7.21 (t, 1H, J=7.9 Hz), 6.99-6.92 (m, 2H), 6.85-6.79 (m, 2H), 6.74-6.66 (m, 2H), 4.00 (t, 2H, J=6.4 Hz), 3.20-3.12 (m, 4H), 2.69-2.62 (m, 4H), 2.48 (m, 2H, J=7.7 Hz), 1.89-1.79 (m, 2H), 1.79-1.68 (m, 2H). MS: 508 [M+H⁺].

### Compound 71. 1-(Benzenesulfonyl)-4-[4-[4-(2-isopropoxyphenoxy)butyl]-piperazin-1-yl]indole

The title compound was prepared starting from amine (IIA-6) and halogen derivative (III-29) according to the procedure a2).

¹H-NMR (300 MHz, CDCl₃): 7.89-7.84 (m, 2H), 7.64 (d, 1H, J=8.4 Hz), 7.55-7.48 (m, 2H), 7.45-7.38 (m, 2H), 7.21 (t, 1H, J=7.9 Hz), 6.99-6.92 (m, 2H), 6.85-6.79 (m, 2H), 6.74-6.66 (m, 2H), 4.50-4.40 (m, 1H), 4.01 (t, 2H, J=6.4 Hz), 3.40-3.20 (m, 4H), 2.70-2.60 (m, 4H), 2.50 (t, 2H, J=7.9 Hz), 1.90-1.80 (m, 2H), 1.47 (d, 9H, J=4.6 Hz). MS: 548 [M+H⁺].

### Compound 72. 2-[3-[4-[1-(Benzenesulfonyl)indol-4-yl]piperazin-1-yl]propoxy]-benzamide

The title compound was prepared starting from amine (IIA-6) and halogen derivative (III-30) according to the procedure a1).

¹H-NMR (300 MHz, CDCl₃): δ 8.20 (dd, 2H, J=1.8 and 7.9 Hz), 7.91-7.88 (m, 1H), 7.50-7.41 (m, 4H), 7.10-6.94 (m, 6H), 6.61 (d, 1H, J=6.9 Hz), 6.53 (dd, 1H, J=0.7 and 3.0 Hz), 5.92-5.87 (m, 1H), 5.23 (s, 2H), 4.24 (t, 2H, J=6.4 Hz), 3.32-3.29 (m, 4H), 2.77-2.70 (m, 4H), 2.61 (t, 2H, J=7.1 Hz), 2.15-2.08 (m, 2H). MS: 520 [M+H⁺].

### Compound 73. 2-[4-[4-[1-(Benzenesulfonyl)indol-4-yl]piperazin-1-yl]butoxy]-benzamide

The title compound was prepared starting from amine (IIA-6) and halogen derivative (III-31) according to the procedure a1).

¹H-NMR (300 MHz, CDCl₃): δ 8.20 (dd, 2H, J=1.8 and 7.7 Hz), 7.86 (d, 2H, J=7.6 Hz), 7.82-7.78 (m, 1H), 7.64 (d, 1H, J=8.4 Hz), 7.54-7.38 (m, 5H), 7.26-7.18 (m, 2H), 7.04 (t, 1H, J=7.6 Hz), 6.98 (d, 1H, J=8.4 Hz), 6.72-6.84 (m, 1H), 4.18 (t, 2H, J=6.4 Hz), 3.15-3.10 (m, 4H), 2.68-2.60 (m, 4H), 2.50 (t, 2H, J=6.9 Hz), 1.90-1.80 (m, 2H), 1.78-1.70 (m, 2H). MS: 532 [M+H⁺].

### EXAMPLE 3

### In Vitro Pharmacology: Binding Assays

The affinity of compounds of the present invention to dopaminergic, serotoninergic, adrenergic, muscarinic M3, histaminergic H1, sigma and serotonine trensporter SERT receptors was tested using the methods as described below, by measurement their binding to these receptors using radioreceptors methods. Moreover, ability of the compounds of the invention to block potassium channel hERG was tested.

The specific ligand binding to the receptors is defined as the difference between the total binding and the non-specific binding determined in the presence of an excess of unlabelled ligand.

The results are expressed as a percent of control specific binding ((measured specific binding/control specific binding)×100%) and as Kᵢ values (inhibition constant). The compounds were tested for their affinity to receptors at a concentration of 1×10⁻⁶ M, and for ability to block potassium channel hERG at a concentration of 1×10⁻⁵ M.

The inhibition constants (Kᵢ) were calculated using the Cheng Prusoff equation (Kᵢ=IC₅₀/(1+(L/K_{D})), where L=concentration of radioligand in the assay, and K_{D}=affinity of the radioligand for the receptor). A Scatchard plot is used to determine the Kd. The IC₅₀ values (concentration causing a half-maximal inhibition of control specific binding) and Hill coefficients (nH) were determined by non-linear regression analysis of the competition curves generated with mean replicate values using Hill equation curve fitting (Y=D+[(A−D)/(1+(C/C₅₀)^{nH})], where Y=specific binding, D=minimum specific binding, A=maximum specific binding, C=compound concentration, C₅₀=IC₅₀, and nH=slope factor). This analysis was performed using a software developed at Cerep (Hill software) and validated by comparison with data generated by the commercial software SigmaPlot® 4.0 for Windows® (©1997 by SPSS Inc.).

Conditions and methodology of in vitro tests are given by reference to the literature.

### Affinity for Dopaminergic Receptors D2 and D3

Experimental conditions for tests are given in Table 1, the results of tests for so representative compounds are given in Table 2a and 2b (receptors D2 and D3) and in Table 3 (receptors D4).

TABLE 1
Experimental conditions for testing the affinity for dopaminergic receptors

| | D2 | D3 | D4 |
|---|---|---|---|
| Biological | human recombinant | human recombinant D3 | human recombinant |
| material | (Invitrogen, GeneBLAzer ® | receptors (Membrane | (CHO cells) |
| | D2-Gqo5 CHO-K1 DA) | Target Systems ™ human | |
| | | dopamine D3 Receptor, | |
| | | PerkinElmer) | |
| Radioligand | [3H]methylspiperone | [3H]methylspiperone | [³H]methylspiperone |
| Concentration | about 0.55 nM | 0.3 nM | 0.3 nM |
| Kd | 0.4 nM | 0.1 nM | 0.19 nM |
| Non-specific | haloperidol (1 μM) | (+)-butactamol (1 μM) | (+)-butaclamol (10 μM) |
| binding | | | |
| Incubation | 60 min, 30° C. | 60 min, 24° C. | 60 min, 22° C. |
| Methodology | Bryan L. Roth. Assay | Missale et al. (1998), | Van Tol, H. H. M et |
| | Protocol Book. University of | Physiol. Rev., 78: 189- | al. (1992) Nature, |
| | North Carolina At Chapel | 225 | 358: 149-152 |
| | Hill. National Institute of | | |
| | Mental Health. | | |
| | Psychoactive Drug | | |
| | Screening Program. | | |
| | Available on-line at | | |
| | 31.08.2008: | | |
| | http://pdsp.med.unc.edu/ | | |
| | UNC- | | |
| | CH%20Protocol%20Book.pdf | | |

TABLE 2a
Results of binding assays for receptors D2 and D3
for representative compounds of the invention

| Compound | D2 | D3 |
|---|---|---|
| No. | [%] | [%] |
| 4 | 96 | 100 |
| 5 | 98 | 101 |
| 12 | 91 | 95 |
| 15 | 93 | 93 |
| 21 | 90 | 86 |
| 23 | 91 | 98 |
| 24 | 97 | 100 |
| 25 | 93 | 85 |
| 60 | 86 | 89 |
| 61 | 96 | 98 |
| 62 | 87 | 101 |
| 63 | 101 | 97 |
| 64 | 86 | 101 |
| 65 | 97 | 100 |
| 66 | 102 | 100 |
| 67 | 99 | 97 |
| 68 | 82 | 100 |
| 69 | 64 | 99 |
| 70 | 63 | 99 |
| 71 | 76 | 98 |
| 72 | 97 | 96 |

TABLE 2a
Inhibition constants Kᵢ for receptors D2 and D3
for representative compounds of the invention

| Compound | D2 | D3 |
|---|---|---|
| No. | [nM] | [nM] |
| 23 | 32.0 | 3.4 |
| 24 | 1.3 | 0.81 |
| 67 | 1.3 | — |
| 72 | 3.2 | — |

TABLE 3
Results of binding assay for receptors D4.4
for representative compounds of the invention

| Compound | D4.4 |
|---|---|
| No. | [%] |
| 4 | 63 |
| 5 | 40 |
| 23 | 18 |
| 24 | 42 |
| 63 | 46 |
| 67 | 44 |
| 72 | 95 |

### Affinity for Serotoninergic Receptors 5-HT1A, 5-HT2A, 5-HT6, 5-HT7 and 5-HT2C

Experimental conditions for tests are given in Table 4, and results of tests for representative compounds of the invention are given in Table 5a and 5b (receptors 5-HT1A, 5-HT2A, 5-HT6 and 5-HT7) and in Table 6a and 6b (receptors 5-HT2C).

TABLE 4
Experimental conditions for testing the affinity for serotoninergic receptors

| | 5-HT1A | 5-HT2A | 5-HT2C | 5-HT6 | 5-HT7 |
|---|---|---|---|---|---|
| Biological | rat | human | human recombinant | human | human |
| material | hippocampus | recombinant | (HEK-293 cells) | recombinant | recombinant |
| | | (Membrane | | (Membrane | (Membrane |
| | | Target Systems ™ | | Target Systems ™ | Target |
| | | Human Serotonin | | human | Systems ™ |
| | | 5-HT2A Receptor, | | Serotonin 5-HT6 | human |
| | | PerkinElmer) | | Receptor, | Serotonin 5- |
| | | | | PerkinElmer) | HT7 Receptor, |
| | | | | | PerkinElmer) |
| Radioligand | [3H]8-OH-DPAT | [3H]ketanserin | [³H]mesulergine | [3H]LSD | [3H]LSD |
| Concentration | 0.8-1.0 nM | 1 nM | 1 nM | 2.5 nM | 3 nM |
| Kd | 1.0 nM | 0.95 nM | 0.5 nM | 1.9 nM | 2.6 nM |
| Non-specific | serotonin | mianserin | RS 102221 | methiothepin | methiothepin |
| binding | (1 μM) | (1 μM) | (10 μM) | (1 μM) | (1 μM) |
| Incubation | 20 min, 37° C. | 60 min, 30° C. | 120 min, 37° C. | 60 min, 30° C. | 120 min, 30° C. |

Methodology:
5-HT1A: Borsini et al. (1995), Naunyn. Sch. Arch. Pharmacol. 352: 276-282
5-HT2A : Bryan L. Roth. Assay Protocol Book. University of North Carolina At Chapel Hill. National Institute of Mental Health. Psychoactive Drug Screening Program. Available on-line at 31.08.2008: http://pdsp.med.unc.edu/UNC-CH%20Protocol%20Book.pdf
5-HT2C: Stam et al. (1994), Eur. J. Pharmacol., 269: 339-348
5-HT6: Bryan L. Roth. Assay Protocol Book. University of North Carolina At Chapel Hill. National Institute of Mental Health. Psychoactive Drug Screening Program. Available on-line at 31.08.2008: http://pdsp.med.unc.edu/UNC-CH%20Protocol%20Book.pdf
5-HT7: Bryan L. Roth. Assay Protocol Book. University of North Carolina At Chapel Hill. National Institute of Mental Health. Psychoactive Drug Screening Program. Available on-line at 31.08.2008: http://pdsp.med.unc.edu/UNC-CH%20Protocol%20Book.pdf

TABLE 5a
Results of binding assays for serotoninergic receptors
for representative compounds of the invention

| Compound | 5-HT1A | 5-HT2A | 5-HT6 | 5-HT7 |
|---|---|---|---|---|
| No. | [%] | [%] | [%] | [%] |
| 4 | 99 | 99 | 98 | 71 |
| 5 | 100 | 100 | 99 | 79 |
| 12 | 85 | 87 | 97 | 70 |
| 15 | 90 | 86 | 96 | 80 |
| 21 | 91 | 80 | 97 | 78 |
| 23 | 97 | 73 | 101 | 19 |
| 24 | 100 | 99 | 101 | 77 |
| 25 | 86 | 86 | 99 | 25 |
| 60 | 46 | 104 | 93 | 62 |
| 61 | 87 | 108 | 91 | 55 |
| 62 | 62 | 103 | 89 | 66 |
| 63 | 99 | 100 | 101 | 69 |
| 64 | 65 | 103 | 96 | 67 |
| 65 | 87 | 104 | 87 | 58 |
| 66 | 78 | 103 | 91 | 70 |
| 67 | 95 | 99 | 99 | 66 |
| 68 | 74 | 104 | 96 | 72 |
| 69 | 88 | 97 | 100 | 53 |
| 70 | 80 | 96 | 99 | 65 |
| 71 | 31 | 89 | 103 | 67 |
| 72 | 98 | 98 | 98 | 58 |

TABLE 5b
Inhibition constants Kᵢ for serotoninergic receptors
for representative compounds of the invention

| Compound | 5-HT1A | 5-HT2A | 5-HT6 | 5-HT7 |
|---|---|---|---|---|
| No. | [nM] | [nM] | [nM] | [nM] |
| 23 | 6.2 | 170.0 | 2.1 | 1 500.0 |
| 24 | 1.2 | 3.2 | 0.35 | 67.0 |
| 67 | 7.0 | 2.4 | 7.3 | — |
| 72 | 3.9 | 18.0 | 0.31 | — |

TABLE 6a
Results of binding assays for serotoninergic 5-HT2C receptors
for representative compounds of the invention

| Compound | 5-HT2C |
|---|---|
| No. | [%] |
| 4 | 73 |
| 5 | 86 |
| 23 | 0 |
| 24 | 48 |
| 63 | 89 |
| 67 | 76 |
| 72 | 35 |

TABLE 6b
Inhibition constants Ki for 5-HT2C serotoninergic receptors
for representative compounds of the invention

| Compound | 5-HT2C |
|---|---|
| No. | [nM] |
| 23 | 10 000.0 |
| 24 | 610.0 |

### Affinity for Adrenergic α1 and α2C Receptors

Experimental conditions for tests are given in Table 7, and results of tests for representative compounds are given in Tables 8a and 8b (α1 receptors) and in Tables 9a and 9b (α2C receptors).

TABLE 7
Experimental conditions for testing
the affinity for adrenergic receptors

| | α1 | α2C |
|---|---|---|
| Biological | rat cerebral | human recombinant |
| material | cortex | (CHO cells) |
| Radioligand | [3H]prazosina | [³H]RX 821002 |
| [symbol] | 0.2 nM | 2 nM |
| Kd | 0.2 nM | 0.95 nM |
| [symbol] | Risperidon (1 μM) | (−)epinefryna |
| niespecyficzne | | (100 μM) |
| Inkubacja | 30 min, 30° C | 60 min, 22° C. |
| Methodology | Leopoldo M et al. | Devedjian et al. |
| | (2002), J Med Chem., | (1994), Eur. J. |
| | (26): 5727-35 | Pharmacol., |
| | | 252: 43-49 |

TABLE 8a
Results of test of affinity for α1 adrenergic receptors for representative
compounds of the invention

| Compound No. | α1 [%] |
|---|---|
| 4 | 68 |
| 5 | 88 |
| 12 | 83 |
| 15 | 93 |
| 21 | 83 |
| 23 | 55 |
| 24 | 92 |
| 25 | 25 |
| 60 | 56 |
| 61 | 13 |
| 62 | 50 |
| 63 | 86 |
| 64 | 57 |
| 65 | 59 |
| 66 | 42 |
| 67 | 70 |
| 68 | 56 |
| 69 | 49 |
| 70 | 54 |
| 71 | 40 |
| 72 | 87 |

TABLE 8b
Inhibition constants Ki for α1 adrenergic receptors for representative
compounds of the invention

| Compound No. | α1 [nM] |
|---|---|
| 23 | 290.0 |
| 24 | 22.0 |

TABLE 9a
Results of test of affinity for α2C adrenergic receptors for
representative compounds of the invention

| Compound No. | α2C [%] |
|---|---|
| 4 | 93 |
| 5 | 93 |
| 23 | 32 |
| 24 | 78 |
| 63 | 100 |
| 67 | 97 |
| 72 | 74 |

TABLE 9b
Inhibition constants Ki for α2C adrenergic receptors for representative
compounds of the invention

| Compound No. | α2C [nM] |
|---|---|
| 23 | 440.0 |
| 24 | 150.0 |

### Affinity for Muscarinic Receptors

Experimental conditions for tests are given in Table 10, and results of tests for representative compounds are given in Table 11.

TABLE 10
Experimental conditions for testing the affinity
for M3 muscarinic receptors

| | M3 |
|---|---|
| Biological material | human recombinant, (CHO cells) |
| Radioligand | [³H]4-DAMP |
| Concentration | 0.2 nM |
| Kd | 0.5 nM |
| Non-specific binding | atropine (1 μM) |
| Incubation | 60 min, 22° C. |
| Methodology | Peralta et al. (1987), Embo. J., 6: 3923-3929. |

TABLE 11
Results of test of affinity for M3 muscarinic receptors for
representative compounds of the invention

| Compound No. | M3 [%] |
|---|---|
| 23 | 16 |
| 24 | 37 |
| 72 | 23 |

### Affinity for Serotonine Transporter (SERT) Receptor

Experimental conditions for tests are given in Table 12, and results of tests for representative compounds are given in Table 13.

TABLE 12
Experimental conditions for testing the affinity for
serotonine transporter (SERT) receptor

| | SERT |
|---|---|
| Biological material | human recombinant SERT receptor (CHO cells) |
| Radioligand | [³H]imipramine |
| Concentration | 2 nM |
| Kd | 1.7 nM |
| Non-specific binding | imipramine (10 μM) |
| Incubation | 60 min, 22° C. |
| Methodology | Tatsumi et al. (1999), Eur. J. Pharmacol., 368: |
| | 277-283. |

TABLE 13
Results of serotonine transporter (SERT) receptor affinity tests for
representative compounds of the invention

| Compound No. | SERT [%] |
|---|---|
| 4 | 79 |
| 5 | 89 |
| 12 | 14 |
| 15 | |
| 21 | 13 |
| 23 | −3 |
| 24 | 23 |
| 25 | 0 |
| 60 | 62 |
| 61 | 55 |
| 62 | 66 |
| 63 | 69 |
| 64 | 67 |
| 65 | 58 |
| 66 | 70 |
| 67 | 66 |
| 68 | 72 |
| 69 | 53 |
| 70 | 65 |
| 71 | 67 |
| 72 | 58 |

Affinity for H1 Histaminergic and σ Receptors

Experimental conditions for tests are given in Table 14, and results of tests for representative compounds are given in Table 15a and 15b.

TABLE 14
Experimental conditions for testing the affinity for H1
histaminergic and σ receptors

| | σ | H1 |
|---|---|---|
| Biological | rat cerebral cortex | human recombinant |
| material | | (HEK-293 cells) |
| Radioligand | [³H]DTG | [³H]pyrilamine |
| Concentration | 8 nM | 1 nM |
| Kd | 29 nM | 1.7 nM |
| Non-specific | haloperidol (10 μM) | pyrilamine (1 μM) |
| binding | | |
| Incubation | 120 min, 22° C. | 60 min, 22° C. |
| Methodology | Shirayama et al. (1993), | Smit et al. (1996), Brit. J. |
| | Eur. J. Pharmacol., 237: | Pharmacol., 117: |
| | 117-126 | 1071-1080. |

TABLE 15a
Results of σ and H1 receptors affinity tests for representative
compounds of the invention

| Compound No. | σ [%] | H1 [%] |
|---|---|---|
| 4 | 45 | 73 |
| 5 | 52 | 88 |
| 23 | 13 | 72 |
| 24 | 57 | 78 |
| 63 | — | 81 |
| 72 | — | 80 |

TABLE 15b
Inhibition constants Ki for H1 histaminergic receptors for
representative compounds of the invention

| Compound No. | H1 [nM] |
|---|---|
| 23 | 150.0 |
| 24 | 100.0 |

Ability to Block hERG Potassium Channel

Ability to block hERG potassium channels was determined using the electrophysiological method and cloned hERG potassium channels (KCNH₂ gene, expressed in CHO cells) as biological material. The effects were evaluated using IonWorks™ Quattro system (MDS-AT).

hERG current was elicited using a pulse pattern with fixed amplitudes (conditioning prepulse: −80 mV for 25 ms; test pulse: +40 mV for 80 ms) from a holding potential of 0 mV. hERG current was measured as a difference between the peak current at 1 ms after the test step to +40 mV and the steady-state current at the end of the step to +40 mV.

Data acquisition and analyses were performed using the IonWorks Quattro™ system operation software (version 2.0.2; Molecular Devices Corporation, Union City, Calif.). Data were corrected for leak current.

The hERG block was calculated as: % Block=(1−ITA/IControl)×100%, where IControl and ITA were the currents elicited by the test pulse in control and in the presence of a test compound, respectively.

TABLE 16a
Results of hERG potassium channels affinity tests
for representative compounds

| Compound no. | hERG [%] |
|---|---|
| 4 | 27 |
| 5 | 30 |
| 23 | 7 |
| 24 | 9 |

TABLE 16b
Inhibition constants Ki for hERG potassium channels
for representative compounds

| Compound no. | H1 [nM] |
|---|---|
| 23 | >10 000 |
| 24 | >10 000 |

Results of in vitro tests as presented above show that compounds of the invention display high affinity for D2, D3,5-HT1A, 5-HT2A, 5-HT6, 5-HT7, alpha1 and alpha2c receptors. This confirms their potential usefulness in the treatment of diseases connected with disturbances in dopaminergic, serotoninergic and noradrenergic transmission, e.g. psychoses, depression as well as anxiety disorders etc. It should be stressed that some of the compounds possess simultaneously high affinity for D2, D3 and for 5-HT1A and/or 5-HT6 receptors, as well as for SERT receptors. Such a pharmacological profile suggests possible efficacy in the treatment of psychoses as well as antidepressant, precognitive and mood stabilizing activity. At the same time compounds of the invention possess weak affinity for hERG potassium channel and M3 muscarinic receptor, and moderate affinity for H1 and 5-HT2C receptors. This may potentially contribute to lack of side effects such as cardiac arrhythmia, vegetative disorders, excessive appetite or metabolic disorders, which may be caused by drugs currently used in therapy of the above-mentioned diseases.

### EXAMPLE 4

### In Vitro Pharmacology: Cellular Functional Assays

Conditions and methodology (by reference to the literature) of cellular functional assays are given in Table 17 and the tests results for representative compounds of the invention are presented in Tables 18a and 18b.

TABLE 17
Conditions and methodology of in vitro tests for cellular functional assays

| | | | | Reaction | | |
|---|---|---|---|---|---|---|
| Assay | Origin | Stimulus | Incubation | product | method of detection | Literature |
| D2S (h) | human recombinant, | none (3 μM dopamine for | 28° C. | impedance | cellular dielectric | Payne et al. (2002), J. |
| (agonism) | (HEK-293 cells) | control) | | | spectroscopy | Neurochem., 82: 1106-1117 |
| D2S (h) | human recombinant, | dopamine (30 nM) | 28° C. | impedance | cellular dielectric | Payne et al. (2002), J. |
| (antagonism) | (HEK-293 cells) | | | | spectroscopy | Neurochem., 82: 1106-1117 |
| D3 (h) | human recombinant, | none (0.3 μM dopamine for | 10 min. | cAMP | HTRF | Missale et al. (1998), |
| (agonism) | (CHO cells) | control) | 37° C. | | (Homogenous | Physiol. Rev., 78: 189-225 |
| | | | | | Time Resolved | |
| | | | | | Fluorescence) | |
| D3 (h) | human recombinant, | dopamina (10 nM) | 10 min. | cAMP | HTRF | Missale et al. (1998), |
| (antagonism) | (CHO cells) | | 37° C. | | | Physiol. Rev., 78: 189-225 |
| 5-HT1A (h) | human recombinant, | none (100 nM 8-OHDPAT | 30 min. | cAMP | HTRF | Newman-tancredi et al. |
| (agonism) | (CHO cells) | for control) | 22° C. | | | (2001), Brit. J. Pharmacol., |
| | | | | | | 132: 518-524 |
| 5-HT1A (h) | human recombinant, | 8-OH-DPAT (10 nM) | 30 min. | cAMP | HTRF | Newman-tancredi et al. |
| (antagonism) | (CHO cells) | | 22° C. | | | (2001), Brit. J. Pharmacol., |
| | | | | | | 132: 518-524 |
| 5-HT2A (h) | human recombinant, | none (10 μM serotonin for | 30 min. | IP1 | HTRF | Porter et al. (1999), Brit. J. |
| (agonism) | (HEK-293 cells) | control) | 37° C. | | | Pharmacol., 128: 13-20 |
| 5-HT2A (h) | human recombinant, | serotonin (100 nM) | 30 min. | IP1 | HTRF | Porter et al. (1999), Brit. J. |
| (antagonism) | (HEK-293 cells) | | 37° C. | | | Pharmacol., 128: 13-20 |
| 5-HT6 (h) | human recombinant, | none (10 μM serotonin for | 45 min. | cAMP | HTRF | Kohen et al. (1996), J. |
| (agonism) | (CHO cells) | control) | 37° C. | | | Neurochem., 66: 47-56 |
| 5-HT6 (h) | human recombinant, | serotonin (100 nM) | 45 min. | cAMP | HTRF | Kohen et al. (1996), J. |
| (antagonism) | (CHO cells) | | 37° C. | | | Neurochem., 66: 47-56 |
| 5-HT7 (h) | human recombinant, | none (10 μM serotonin for | 45 min. | cAMP | HTRF | Adham et al. (1998), J. |
| (agonism) | (CHO cells) | control) | 37° C. | | | Pharmacol. Exp. Ther., |
| | | | | | | 287: 508-514 |
| 5-HT7 (h) | human recombinant, | serotonin (300 nM) | 45 min. | cAMP | HTRF | Adham et al. (1998), J. |
| (antagonism) | (CHO cells) | | 37° C. | | | Pharmacol. Exp. Ther. |
| | | | | | | , 287: 508-514 |

The results are expressed as a percent of control specific agonist response ((measured specific response/control specific agonist response)×100) obtained in the presence of tested compound.

The results are expressed as a percent of control specific binding ((measured specific binding/control specific binding)×100%) and as Kb values (dissociation constant). The compounds were tested for affinity thereof to receptors at a concentration of 1×10⁻⁶ M.

The EC50 values (concentration producing a half-maximal specific response) and IC50 values (concentration causing a half-maximal inhibition of the control specific agonist response) were determined by non-linear regression analysis of the concentration-response curves generated with mean replicate values using Hill equation curve fitting (Y=D+[(A−D)/(1+(C/C50)nH)], where Y=specific response, D=minimum specific response, A=maximum specific response, C=compound concentration, and C50=EC50 or IC50, and nH=slope factor).

This analysis was performed using a software developed at Cerep (Hill software) and validated by comparison with data generated by the commercial software SigmaPlot® 4.0 for Windows® (© 1997 by SPSS Inc.).

For the antagonists, the apparent dissociation constants (Kb) were calculated using the modified Cheng Prusoff equation (Kb=IC50/(1+(A/EC50A)), where A=concentration of reference agonist in the assay, and EC50A=EC50 value of the reference agonist).

TABLE 18a
Results of cellular functional assays for representative compounds

| Compound | | D2-antag | | D3-antag | 5-HT1A-ag | 5-HT1A- | 5-HT2A-ag | 5-HT2A- | 5-HT6-ag | 5-HT6- |
|---|---|---|---|---|---|---|---|---|---|---|
| no. | D2-ag [%] | [%] | D3-ag [%] | [%] | [%] | antag [%] | [%] | antag [%] | [%] | antag [%] |
| 4 | 45 | 89 | 22 | 29 | 63 | 17 | 2 | 17 | 0 | 73 |
| 5 | 47 | 99 | 22 | 43 | 66 | 31 | 8 | 66 | −1 | 42 |
| 12 | 11 | 73 | — | — | — | — | — | — | −2 | 73 |
| 15 | 11 | 68 | — | — | — | — | — | — | 0 | 88 |
| 23 | 40 | 55 | 65 | −19 | 69 | 20 | — | — | 2 | 42 |
| 24 | 47 | 93 | 13 | 63.2 | 69.7 | 0 | 0 | 34.2 | 0 | 88.7 |
| 25 | 30 | 75 | — | — | — | — | — | — | −4 | 98 |
| 63 | 47 | 108 | 20 | 72 | 26 | 73 | 6 | 63 | −1 | 61 |
| 67 | 46 | 103 | 11 | 74 | 23 | 56 | 4 | 34 | 0 | 53 |
| 72 | 34 | 95 | 34 | 74 | 16 | 61 | 2 | 45 | −2 | 103 |

TABLE 18b
Inhibition constants Ki for the representative compound

| | |
|---|---|
| agonism | antagonism |

| | |
|---|---|
| Receptor | Ki (nM) |

| | | |
|---|---|---|
| D2 | 350 | 55 |
| D3 | 98 | 290 |
| 5-HT1A | 180 | N.C. |
| 5-HT2A | N.C. | 1 700 |
| 5-HT6 | N.C. | 66 |

N.C.—not calculable

The representative compound showed a unique cellular functional profile by combining the partial agonism at dopamine D2 receptors and antagonism at serotonin 5-HT6 receptors. Moreover, the compound demonstrated the beneficial agonistic properties at 5-HT1A receptors, partially agonistic properties at D3 receptors and antagonistic properties at 5-HT2A receptors. Such properties indicate the possibility to combine activity, which is beneficial from the point of view of antipsychotic effect and depends on modulation of dopaminergic system with pro-cognitive, anxiolytic and antidepressant activity.

### EXAMPLE 5

### Behavioral Tests in Mice

### Antipsychotic Activity in Mice

Potential antipsychotic activity was tested for the representative compound 24 in mouse model of psychosis, involving the induction of locomotor hyperactivity by administering psychotomimetic substance-dizocilpine. The ability of a test compound to remove this effect is a measure of potential antipsychotic activity.

### Animals

Male CD-1 mice were group-housed for 2-3 day period in polycarbonate Makrolon type 3 cages (dimensions 26.5×15×42 cm) in an environmentally controlled, experimental room (ambient temperature 22-20° C.; relative humidity 50-60%; 12:12 light:dark cycle, lights on at 8:00), in groups of 15. Standard laboratory food (Ssniff M-Z) and filtered water were freely available. On the day before experiments the equipment produced “white noise” was turned on for 30 minutes and mice were weighted exact to 1 g. Animals were assigned randomly to treatment groups. All the experiments were performed by two observers unaware of the treatment applied between 9:00 and 14:00 on separate groups of animals. Mice were used only once and were killed immediately after the experiment.

### Dizocilpine-Induced Locomotor Hyperactivity

The locomotor activity was recorded with an Opto M3 multi-channel activity monitor (MultiDevice Software v.1.3, Columbus Instruments). The mice were individually placed in plastic cages (22×12×13 cm) for 30 minutes habituation period, and then the crossings of each channel (ambulation) were counted during 1 h with data recording every 5 minutes. The cages were cleaned up with 70% ethanol after examining each mouse. Drugs were administered to 10 mice per treatment group. Test compounds were given 30 minutes before the experiment. Dizocilpine was administered 30 minutes before the test.

### Test Compounds

Test compounds were prepared as a suspension in 1% aqueous solution of Tween 80, and dizocilpine was dissolved in distilled water immediately before administration. An injection volume of 10 ml/kg was used and all compounds were administered intraperitoneally (i.p.).

### Tail Suspension Test in C57BL/6J Mice

Within 24 h prior to testing, animals were habituated to experimental conditions. For this purpose mice in home cages were transferred to the experimental room for 15 min., while maintaining the lighting and the “white noise” characteristic for the experiment.

The testing procedure was performed on male C57BL/6J mice and based on a method of Steru et al. (The tail suspension test: a new method for screening antidepressants in mice, Psychopharmacology 85, 367-370, 1985). An automated device (Kinder Scientific) was used. After 1 h of adaptation in the experimental room with low light, the mice received the test compound intraperitoneally (at least 3 selected doses).

After a specified time after administration of the test compound mice were suspended by the tail with tape to an aluminum hook connected to a strain gauge. Mice were positioned such that the base of their tail was aligned with the bottom of the hook. This positioning was found to decrease the propensity for mice to climb their tail during the test. A strain gauge connected to computer software detected any movements by the mouse in order to record the number of times (events) each subject enters into an escape behavior (struggling episodes), the duration of the event, and the average strength of each event during a 6-min test session. The following settings were used in all experiments: threshold 0.20 Newtons, off delay 30 msec.

### Four-Plate Test in Swiss Albino Mice

Within 24 h prior to testing, animals were habituated to experimental conditions. For this purpose mice in home cages were transferred to the experimental room for 15 min., while maintaining the lighting and the “white noise” characteristic for the experiment.

The four-plate test (BIOSEB, France) was performed in a cage (25×18×16 cm) floored by four identical rectangular metal plates (8×11 cm) separated from one another by a gap of 4 mm. The top of the cage was covered by a transparent Perspex lid that prevented escape behaviour. The plates were connected to a device that generated electric shocks. Animals were placed individually in the experimental cage and following a 15-s habituation period, the animal's motivation to explore a novel environment was suppressed by an electric foot shock (0.8 mA, 0.5 s) every time it moved from one plate is to another during a 1-minute test session. This action is referred to as a ‘punished crossing’, and was followed by a 3 s shock interval, during which the animal could move across plates without receiving a shock. The measure of anxiolytic activity of the compound was the number of ‘punished crossings’ from one plate to the adjacent during 1-min test period.

### Behavioral Tests in Rats

### Animals

Animals for these tests were prepared as follows:

Drug-naive male Wistar rats (Charles River, Sulzfeld, Germany) weighing 250-400 g were housed in polycarbonate Makrolon cages (380×200×590 mm) in an environmentally controlled, experimental room (ambient temperature 21-23° C.; relative humidity 50-60%; 12:12 light:dark cycle, lights on at 7:00 a.m.), in groups of 4. Tap water and standard lab chow (Labofeed H, WPIK, Kcynia, Poland) was available ad libitum.

The animals were delivered to the Animal Research Unit of the Department of Pharmacology, Institute of Psychiatry and Neurology, 2 weeks before the start of experimental procedures. During these 2 weeks all the rats were repeatedly getting used to the presence of the experimenter (handling), and to injections of saline.

On the day before experiments rats were weighted exact to 1 g. Animals were assigned randomly to treatment groups. All the experiments were performed on separate groups of animals, between 9:00 and 15:00. Tested compound was administered intraperitoneally (i.p.) in an injection volume of 2 ml/kg in at least 3 selected doses. Control groups received an appropriate vehiculum.

All animals were used only once and were euthanized immediately after the experiment.

### Conflict Test (Vogel Test) in Rats

24 Hours before the experiment animals were habituated to test conditions. For this purpose rats in home cages were transferred to the experimental room for 15 min., while maintaining the lighting and the “white noise”, characteristic for the experiment.

The test was performed by modified method described by Vogel et al. (Psychopharmacologia, 21 (1), 8-12, 1971) using the monitoring system TSE Systems. The system consisted of a polycarbonate cage (dimensions 26.5×15×42 cm), equipped with a grid floor made from stainless steel bars and a drinking bottle containing tap water. Experimental chambers (two) were connected to PC software by control chassis and a device that generates electric shocks. The experiment lasted 3 days. On the first day of the experiment, the rats were placed individually in the experimental cage equipped with a drinking bottle and were adapted to the test chamber for 10 min. After the adaptation period, the animals were deprived of water for 24 h and were then placed in the test chamber for another 10-min adaptation period during which they had free access to the drinking bottle. Afterwards, rats were allowed a 1 hour free-drinking session in their home cage. After another 24-h water deprivation period, the rats after administration of the test compound were placed in the test chamber. Recording data started immediately after the first lick and every 20 licks rats were punished with an electric shock (0.5 mA, lasting 1 s). The impulses were released via the spout of the drinking bottle. If a rat was drinking when an impulse was released, it received a shock. The number of licks and the number of shocks received throughout a 5-min experimental session were recorded automatically.

### Reversal of Dizocitpine (MK-801)-Induced PPI Deficits

### Evaluation of Preputse Inhibition (PPI)

The PPI apparatus consisted of eight acoustic startle chambers (SR-LAB, San Diego Instruments, San Diego, Calif., USA). Each chamber consisted of a Plexiglas cylinder (8.9 cm diameter×20 cm long) resting on a Plexiglas frame in a sound attenuated, ventilated enclosure. Background noise and acoustic stimuli were presented via a loudspeaker mounted 24 cm above the animal. Startle responses, reflecting the motion of animals in the cylinder following the acoustic stimulus, were detected by a piezoelectric transducer mounted below the frame. The administration of stimuli and response recording were controlled by the SR-LAB software. Test sessions started with a 5-minutes acclimatization period. Throughout the whole session, the chamber light was on, and the background white noise was set at 70 dB. The test session included 3 initial startling stimuli (intensity: 120 dB, duration: 40 ms) to accustom the rat to the to experimental procedure. The initial stimuli were followed by 60 trials (6×10 trials) presented in a random order:
10 background trials (B) which involved a presentation of a sham stimulus intensity: 70 dB, duration: 40 ms),
two types (2×10) of prepulse trials (PP) which included only a prepulse stimuli (84 dB or 90 dB, 20 ms),
10 pulse trials (P) which included only a pulse startling stimulus (120 dB, 40 ms),
two types (2×10) of prepulse-and-pulse trials (PP-P) which involved a prepulse (84 dB or 90 dB, 20 ms) followed 100 ms later by a 120-dB, 40 ms. pulse stimulus (P).

The average inter-trial interval was 22.5 s (range: 15-30 s). This interval was randomized by the SR-LAB software. Startle responses were measured for 100 ms after the onset of the last trial stimulus. For each type of stimulation, startle amplitudes were averaged across the 10 trials. The magnitude of PPI was calculated as a percent inhibition of the startle amplitude in the pulse trial (treated as 100%) according to the formula: [(startle amplitude in P trials−startle amplitude in PP-P trials)/startle amplitude in P trials]×100%. Startle responses to the 3 initial stimuli of intensity of 120 dB were excluded from the statistical analyses.

### Reversal of the Dizocilpine (MK-801)-Induced PPI Deficits

### Test Compound

Compound 24 was administered intraperitoneally as a suspension in 1% aqueous solution of Tween 80 in an injection volume of 2 ml/kg 60 min. before the test. Dizocilpine was dissolved in saline immediately before administration and administered intraperitoneally in an injection volume of 1 ml/kg 15 min. before the session.

TABLE 19
Results of behavioral tests in animals for the representative
compound of the invention

| Compound 24 | MED* [mg/kg] |
|---|---|
| Dizocilpine-induced locomotor hyperactivity in mice | 10 |
| Tail suspension test in C57BL/6J mice | 1.25 |
| Four-plate test in Swiss albino mice | 5 |
| Conflict test (Vogel test) in rats | 3 |
| Reversal of the dizocilpine (MK-801)-induced PPI | 10 |
| deficits in rats | |

*minimum effective dose

The representative Compound 24 showed a broad psychotropic activity. It was active in dizocilpine-induced locomotor hyperactivity test in mice, demonstrating the potential in the therapy of positive symptoms of schizophrenia, as well as in the dizocilpine (MK-801)-induced PPI deficit test in rats, which is in procedure assessing ability to treat attention deficits and information filtering (dimensions of cognitive deficits), underlying the pathomechanism of schizophrenia. Activity in the prepulse inhibition (PPI) tests is of a special value due to the identity of the modeled disorders in animals and those occurring in humans, and therefore their relatively high translatability on clinical effects, and also due to the low efficiency in removing this type of disturbance by the currently available antipsychotic drugs, especially at doses that cause no adverse effects (Porsolt R. D et al., J. Pharmacol. Exp. Ther., 333(3), 632-8, 2010). The compound 24 was active in well established models for detecting substances with potential antidepressant activity, i.e. tail suspension test in mice as well as potential anxiolytic activity, i.e. four-plate test in mice or conflict drinking test (Vogel) in rats. Such a wide pharmacological activity, beyond the purely antipsychotic effects is a particularly desirable feature of modern antipsychotic drug due to complexity of clinical conditions associated with schizophrenia, including depression and anxiety.

1. Compound of the general formula (IA) 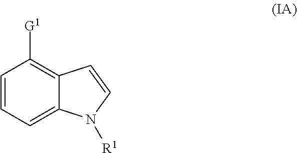
wherein
R¹ represents benzyl unsubstituted or substituted with halogen atom, —OH, or C₁-C₃-alkyl; or phenylsulphonyl unsubstituted or substituted in the phenyl ring with halogen atom, —OH or C₁-C₃-alkyl;
G¹ represents piperazine moiety of the following formula 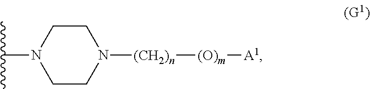
wherein
n is 3 or 4,
m is 1,
A¹ represents phenyl unsubstituted or substituted with one substituent selected from the group consisting of halogen atom, —0H, C₁-C₃-alkyloxy, —CONH₂ and phenyl; a moiety selected from the group consisting of 3,4-dihydroquinolin-2(1H)-on-yl, 1,4-benzodioxanyl and benzofuranyl, which moiety is linked through carbon atom of its benzene ring; or imidazolidin-2-on-yl linked through its nitrogen atom;
and pharmaceutically acceptable salts thereof.

2. The compound according to claim 1, wherein A¹ in G¹ moiety represents phenyl unsubstituted or substituted with one substituent selected from the group consisting of halogen atom, —OH, C₁-C₃-alkyloxy, and phenyl; a moiety selected from the group consisting of 3,4-dihydroquinolin-2(1H)-on-yl, 1,4-benzodioxanyl and benzofuranyl, which moiety is linked through carbon atom of its benzene ring; or imidazolidin-2-on-yl linked through its nitrogen atom.

3. The compounds according to claim 1, wherein A¹ in G¹ moiety represents phenyl unsubstituted or substituted with one substituent selected from the group consisting of halogen atom, —OH, C₁-C₃-alkyloxy, —CONH₂ and phenyl; a moiety selected from the group consisting of 3,4-dihydroquinolin-2(1H)-on-yl, 1,4-benzodioxanyl and benzofuranyl, which moiety is linked through carbon atom of its benzene ring.

4. The compound according to claim 1, wherein A¹ represents 3,4-dihydroquinolin-2(1H)-on-yl.

5. The compound according to claim 1, wherein A¹ represents unsubstituted phenyl.

6. The compound according to 3, wherein A¹ represents phenyl substituted with one substituent selected from the group consisting of halogen atom, —OH, C₁-C₃-alkyloxy, —CONH₂ and phenyl.

7. The compound according to claim 6, wherein the substituent of phenyl ring is selected from the group consisting of halogen atom, C₁-C₃-alkyloxy and —CONH₂.

8. The compound according to claim 1, wherein R¹ represents unsubstituted phenylsulphonyl.

9. The compound according to claim 1, wherein R¹ represents unsubstituted benzyl.

10. The compound according to claim 1, wherein R¹ represents benzyl substituted with halogen atom, preferably with fluorine or chlorine.

11. The compound according to claim 1 selected from the group consisting of the following:
7-[3-[4-(1-benzylindol-4-yl)piperazin-1-yl]propoxy]-3,4-dihydro-1H-quinolin-2-one
7-(4-(4-(1-benzyl-1H-indol-4-yl)piperazin-1-yl)butoxy)-3,4-dihydroquinolin-2(1H)-one
7-[3-[4-[1-[(2-fluorophenyl)methyl]indol-4-yl]piperazin-1-yl]propoxy]-3,4-dihydro-1H-quinolin-2-one
7-[3-[4-[1-[(3-fluorophenyl)methyl]indol-4-yl]piperazin-1-yl]propoxy]-3,4-dihydro-1H-quinolin-2-one
7-[3-[4-[1-[(3-hydroxyphenyl)methyl]indol-4-iyl]piperazin-1-yl]propoxy]-3,4-dihydro-1H-quinolin-2-one
7-[3-[4-[1-(m-tolylmethyl)indol-4-yl]piperazin-1-yl]propoxy]-3,4-dihydro-1H-quinolin-2-one
4-(4-(3-phenoxypropyl)piperazin-1-yl)-1-(phenylsulphonyl)-1H-indole
4-(4-(3-(4-fluorophenoxy)propyl)piperazin-1-yl)-1-(phenylsulphonyl)-1H-indole
4-(4-(3-(2-(1-methylethoxy)phenoxy)propyl)piperazin-1-yl)-1-(phenyl-sulphonyl)-1H-indole
7-[3-[4-[1-(benzensulphonyl)indol-4-yl]piperazin-1-yl]propoxy]-3,4-dihydro-1H-quinolin-2-one
7-(4-(4-(1-(phenylsulphonyl)-1H-indol-4-yl)piperazin-1-yl)butoxy)-3,4-dihydroquinolin-2(1H)-one
4-(4-(3-(benzofuran-6-yloxy)propyl)piperazin-1-yl)-1-(phenylsulphonyl)-1H-indole
2-[3-[4-(1-benzylindol-4-yl)piperazin-1-yl]propoxy]benzamide
7-[4-[4-[1-[(2-fluorophenyl)methyl]indol-4-yl]piperazin-1-yl]butoxy]-3,4-dihydro-1H-quinolin-2-one
2-[3-[4-[1-[(2-fluorophenyl)methyl]indol-4-yl]piperazin-1-yl]propoxy]-benzamide
7-[4-[4-[1-[(3-fluorophenyl)methyl]indol-4-yl]piperazin-1-yl]butoxy]-3,4-dihydro-1H-quinolin-2-one
2-[3-[4-[1-[(3-fluorophenyl)methyl]indol-4-yl]piperazin-1-yl]propoxy]-benzamide
7-[4-[4-[1-[(4-fluorophenyl)methyl]indol-4-yl]piperazin-1-yl]butoxy]-3,4-dihydro-1H-quinolin-2-one
2-[3-[4-[1-[(4-fluorophenyl)methyl]indol-4-yl]piperazin-1-yl]propoxy]-benzamide
7-[4-[4-[1-[(3-chlorophenyl)methyl]indol-4-yl]piperazin-1-yl]butoxy]-3,4-dihydro-1H-quinolin-2-one
2-[3-[4-[1-[(3-chlorophenyl)methyl]indol-4-yl]piperazin-1-yl]propoxy]-benzamide
1-(benzenesulfonyl)-4-[4-(4-phenoxybutyl)piperazin-1-yl]indole
1-(benzenesulfonyl)-4-[4-[4-(4-fluorophenoxy)butyl]piperazin-1-yl]-indole
1-(benzenesulfonyl)-4-[4-[4-(2-isopropoxyphenoxy)butyl]-piperazin-1-yl]indole
2-[3-[4-[1-(benzenesulfonyl)indol-4-yl]piperazin-1-yl]propoxy]-benzamide
2-[4-[4-[1-(benzenesulfonyl)indol-4-yl]piperazin-1-yl]butoxy]-benzamide
and pharmaceutically acceptable salts and solvates thereof.

12. (canceled)

13. A pharmaceutical composition comprising the compound of formula (IA) as defined in claim 1 as an active ingredient in combination with pharmaceutically acceptable carrier(s) and/or excipient(s).

14-15. (canceled)

16. A method of treatment and/or prevention of disorders of the central nervous system related to serotoninergic and dopaminergic transmission in mammals, comprising administration of the pharmaceutically effective amount of the compound of formula (IA) as defined in claim 1 or the pharmaceutical composition as defined in claim 13 wherein the disorder of the central nervous system is selected from schizophrenia; schizoaffective disorders; schizophreniform disorders; delusional syndromes and other psychotic conditions related and not related to taking psychoactive substances; affective disorder; bipolar disorder; mania; depression; anxiety disorders of various etiology; stress reactions; consciousness disorders; coma; delirium of alcoholic or other etiology; aggression; psychomotor agitation and other conduct disorders; sleep disorders of various etiology; withdrawal syndrome of various etiology; addiction; pain syndromes of various etiology; intoxication with psychoactive substances; cerebral circulatory disorders of various etiology; psychosomatic disorders of various etiology; conversion disorders; dissociative disorders; urination disorders; autism and other developmental disorders, including nocturia, stuttering, tics; cognitive disorders of various types, including Alzheimer's disease; psychopathological symptoms and neurological disorders in the course of other diseases of the central and peripheral nervous systems.
